(19) United States
(12) Patent Application Publication
(10) Pub. No.: US 2005/0119226 A1
(43) Pub. Date: Jun. 2, 2005

(54) Methods for synthesizing organoboronic compounds and products thereof

(75) Inventors: Armin Walter, Halle/Westf. (DE); Alfred Olbrich, Halle/Wesf. (DE); Andrea M.T. Weiland-Waibel, Hohenbrunn (DE); Dieter Krimmer, Steinhausen (CH)

Correspondence Address: KLARQUIST SPARKMAN, LLP, 121 SW SALMON STREET, SUITE 1600, PORTLAND, OR, 97204, US

(73) Assignee: Trigen Limited

(21) Appl. No.: 10/937,181

(22) Filed: Sep. 8, 2004

Related U.S. Application Data
(60) Provisional application No. 60/501,718, filed on Sep. 9, 2003.

(51) Int. Cl. C07F 5/02; A61K 31/69

(52) U.S. Cl. 514/64; 562/7

(57) ABSTRACT

Organoboronic acids, for example Cbz-(R)-Phe-(S)-Pro-(R)-Mpg-B(OH)₂, are made by hydrolysing their diethanolamine adducts under conditions which avoid substantial C—B bond breakage. The product acids are substantially free of degradation product derived from cleavage of the C—B bond thereof. The acids are used to make base addition salts thereof. The salts are formulated into anti-thrombotic pharmaceutical formulations.

This application claims the benefit of U.S. Provisional Application No. 60/501,718 filed Sep. 9, 2003, which is incorporated herein by reference.

### BACKGROUND

The present disclosure relates to organoboronic acids and the preparation thereof. The application also relates to the provision of organoboronate salts, to their formulations and to other subject matter.

Organoboronic Acids and Esters

Organoboronic acids and their derivatives have potential utility as pharmaceuticals, particularly as enzyme inhibitors. For a recent review of boronic acids which are potential pharmaceuticals, see Yang et al Medical Research Reviews, 23: 346-368, 2003. The synthesis of organoboronic acids and their derivatives is well documented.

Thus, Matteson D S Chem. Rev. 89: 1535-1551, 1989 reviews the use of α-halo boronic esters as intermediates for the synthesis of inter alia amino boronic acids and their derivatives. Matteson describes the use of pinacol boronic esters in non-chiral synthesis and the use of pinanediol boronic esters for chiral control, including in the synthesis of amino and amido boronate esters.

Organoboronic Acid Stability

Organoboronic acids can be relatively difficult to obtain in analytically pure form. Thus, alkylboronic acids and their boroxines are often air-sensitive. Korcek et al, J. Chem. Soc. Perkin Trans. 2:242, 1972, teaches that butylboronic acid is readily oxidized by air to generate 1-butanol and boric acid.

It is known that derivatisation of boronic acids as cyclic esters provides oxidation resistance. For example, Martichonok V et al J. Am. Chem. Sec. 118:950-958, 1996 state that diethanolamine derivatisation provides protection against possible boronic acid oxidation. U.S. Pat. No. 5,681,978 (Matteson D S et al) teaches that 1,2-diols and 1,3 diols, for example pinacol, form stable cyclic boronic esters that are not easily oxidised.

Wu et al, J. Pharm. Sci., 89: 758-765 (2000), discuss the stability of the compound N-(2-pyrazine)carbonyl-phenylalanine-leucine boronic acid (LDP-341, also known as bortezomib), an anti-cancer agent. It is described how “during an effort to formulate [LDP-341] for parenteral administration, the compound showed erratic stability behaviour”. The degradation pathways were investigated and it was concluded that the degradation was oxidative, the initial oxidation being attributed to peroxides or molecular oxygen and its radicals.

WO 02/059131 discloses boronic acid products which are described as stable. In particular, these products are certain boropeptides and/or boropeptidomimetics in which the boronic acid group has been derivatised with a sugar. The disclosed sugar derivatives, which have hydrophobic amino acid side chains, are of the formula 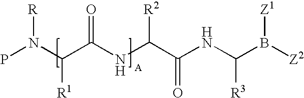
wherein:
P is hydrogen or an amino-group protecting moiety;
R is hydrogen or alkyl;
A is 0, 1 or 2;
R¹, R² and R³ are independently hydrogen, alkyl, cycloalkyl, aryl or —CH₂—R⁵;
R⁵, in each instance, is one of aryl, aralkyl, alkaryl, cycloalkyl, heterocyclyl, heteroaryl, or —W—R⁶, where W is a chalcogen and R⁶ is alkyl;
where the ring portion of any of said aryl, aralkyl, alkaryl, cycloalkyl, heterocyclyl, or heteroaryl in R¹, R², R³ or R⁵ can be optionally substituted; and
Z¹ and Z² together form a moiety derived from a sugar, wherein the atom attached to boron in each case is an oxygen atom.

Some of the disclosed compounds are sugar derivatives of LDP-341 (see above).

Neutral P1 Residue Boropeptide Thrombin Inhibitors

Claeson et al (U.S. Pat. No. 5,574,014 and others) and Kakkar et al (WO 92/07869 and family members including U.S. Pat. No. 5,648,338) disclose lipophilic thrombin inhibitors having a neutral (uncharged) C-terminal (P1) side chain, for example an alkoxyalkyl side chain.

The Claeson et al and Kakkar et al patent families disclose boronate esters containing the amino acid sequence D-Phe-Pro-BoroMpg [(R)-Phe-Pro-BoroMpg], which are highly specific inhibitors of thrombin. Of these compounds may be mentioned in particular Cbz-(R)-Phe-Pro-BoroMpg-OPinacol (also known as TRI 50b). The corresponding free boronic acid is known as TRI 50c. For further information relating to TRI 50b and related compounds, the reader is referred to the following documents:
Elgendy S et al., in The Design of Synthetic Inhibitors of Thrombin, Claeson G et al Eds, Advances in Experimental Medicine, 1993, 340, pp, pp 173-178.
Claeson G et al, Biochem J. 1993, 290, 309-312
Tapparelli C et al, J Biol Chem, 1993, 268, 4734-4741
Claeson G, in The Design of Synthetic Inhibitors of Thrombin, Claeson G et al Eds, Advances in Experimental Medicine, 1993, 340, pp 83-91
Phillip et al, in The Design of Synthetic Inhibitors of Thrombin, Claeson G et al Eds, Advances in Experimental Medicine, 1993, 340, pp 67-77
Tapparelli C et al, Trends Pharmacol. Sci. 1993, 14, 366-376
Claeson G, Blood Coagulation and Fibrinolysis 1994, 5, 411-436
Elgendy et al, Tetrahedron 1994, 50, 3803-3812
Deadman J et al, J. Enzyme Inhibition 1995, 9, 29-41.
Deadman J et al, J. Medicinal Chemistry 1995, 38, 1511-1522.
   Aminoboronate Synthesis

It is known in the prior art to synthesise TRI 50c esters via the following process:

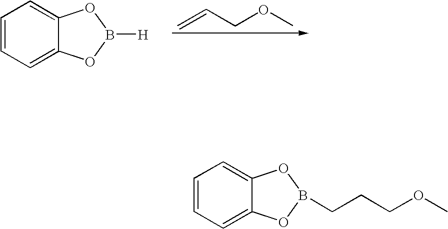

The product of the above step is then converted by known methods to, for example, TRI 50b. See for example Deadman J et al, J. Medicinal Chemistry 1995, 38, 1511-1522.

Base Addition Salts

U.S. Ser. No. 10/659,178, assigned to Trigen Limited, discloses pharmaceutically acceptable base addition salts of boronic acids which have a neutral aminoboronic acid residue capable of binding to the thrombin S1 subsite linked through a peptide linkage to a hydrophobic moiety capable of binding to the thrombin S2 and S3 subsites. In a first embodiment, there is disclosed a pharmaceutically acceptable base addition salt of a boronic acid of, for example, formula (A): 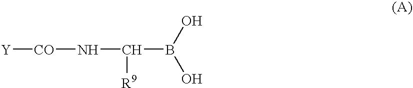
wherein
Y comprises a hydrophobic moiety which, together with the aminoboronic acid residue —NHCH(R⁹)—B(OH)₂, has affinity for the substrate binding site of thrombin; and
R⁹ is a straight chain alkyl group interrupted by one or more ether linkages (e.g. 1 or 2) and in which the total number of oxygen and carbon atoms is 3, 4, 5 or 6 (e.g. 5) or R⁹ is —(CH₂)ₘ—W where m is 2, 3, 4 or 5 (e.g. 4) and W is —OH or halogen (F, Cl, Br or I). R⁹ is an alkoxyalkyl group in one subset of compounds, e.g. alkoxyalkyl containing 4 carbon atoms.

An exemplary boronic acid of formula (A) is TRI 50c.

U.S. Ser. No. 10/659,179, assigned to Trigen Limited, discloses salts of a pharmaceutically acceptable multivalent (at least divalent) metal and an organoboronic acid drug. Such salts are described as having an improved level of stability which cannot be explained or predicted on the basis of known chemistry, and as being indicated to have unexpectedly high and consistent oral bioavailability not susceptible of explanation on the basis of known mechanisms. The oral formulations of such salts are therefore also disclosed.

One particular class of salts comprises those wherein the organoboronic acid comprises a boropeptide or boropeptidomimetic. Such drugs which may beneficially be prepared as salts include without limitation those of the formula X-(aa)ₙ-B(OH)₂, where X is H or an amino-protecting group, n is 2, 3 or 4, (especially 2 or 3) and each aa is independently a hydrophobic amino acid, whether natural or unnatural. In one class of multivalent metal salts, the organoboronic acid is of formula (A) above.

U.S. Ser. No. 10/658,971, assigned to Trigen Limited, discloses and claims inter alia parenteral pharmaceutical formulations that include a pharmaceutically acceptable base addition salt of a boronic acid of, for example, formula (A) above. Such salts are described as having an improved level of stability which cannot be explained or predicted on the basis of known chemistry.

The Examples of U.S. Ser. Nos. 10/659,178, 10/659,179 and 10/658,971 contain data indicating that the stability (resistance to deboronation) of organoboronic acids may be increased by providing them in the form of salts, e.g. metal salts. In single experiments, the ammonium salt of TRI 50c appeared to decompose on drying to yield ammonia, whilst the choline salt demonstrated rapid decomposition to a deboronated impurity. Although experiments have not been conducted to reproduce these unrepeated observations, there is provided a sub-class in which the ammonium and choline salts are excluded. The salt may be an acid salt. In any event, this stabilisation technique forms part of the disclosure and is applicable, inter alia, to organoboronic acids described under the heading “BACKGROUND” and to organoboronic acids described in publications mentioned under that heading.

The ability to manufacture these salts in high purity on a commercially viable scale is an important issue.

### BRIEF SUMMARY OF THE DISCLOSURE

TRI 50c base addition salts are obtained via TRI 50c esters. However, published synthetic routes to TRI 50c esters and thus to TRI 50c give rise to one or more impurities. Original methods for making TRI 50c base addition salts gave rise to one or more impurities and very high purity salts were not obtained. Further, the salts have proved most challenging to obtain in high purity. Thus, purification techniques which were applied failed to produce very high purity salts. HPLC will not be usable on an industrial scale to purify salts made via published TRI 50c ester syntheses and the original salt preparation methods. In other words, in order for the therapeutic benefits of TRI 50c salts to be provided to those in need thereof, the salts must be obtainable industrially in adequately pure form and the pure form must be attainable without the use of excessively expensive purification techniques. It is similarly desirable also for other organoboronates to be available industrially in pure form.

The disclosure relates therefore to organoboronic compounds (organoboronates) and particularly aminoboronates (aminoboronic compounds) and compounds comprising peptide boronate (boronic) moieties.

The disclosure provides techniques for purifying organoboronic compounds and techniques for helping to maintain the purity of organoboronic compounds, and the products of such techniques. The present disclosure further provides a method of making such high purity salts and the high purity salts themselves. In particular, disclosed herein in one embodiment is a method comprising a chirally-selective precipitation step which results in a precipitated boronic acid derivative of high purity. Further provided is a method for hydrolysing organoboronate that can be used to help obtain high purity salts. In another embodiment, there is disclosed a method for preparing the salts described herein in high purity and wherein selected solvents are used to help achieve high purity levels.

In another aspect there is provided a novel synthesis useful in the preparation of the TRI 50c boropeptide and other compounds; also provided are aminoboronates and boropeptides obtainable indirectly from the synthesis.

There are further provided boronic acid salts of specified purity and pharmaceutical formulations containing them.

In one aspect, the disclosure provides the use of diethanolamine to resolve by precipitation boronic acid compounds (whether provided as the acid or, for example, an ester), wherein the acid is of the formula X—(R)-aa¹-(S)-aa²-NH—C*(R¹)H—B(OH)_{2,} where aa¹, aa² and R¹ are as described below and C* is a chiral centre present initially in both chiralities. The disclosure further provides a method of resolving the chiral isomers, in which the diethanolamine is used in an amount of 1.25±0.1 equivalents per equivalent of the boronic acid compound having chiral centre C* in (R)-configuration.

Another aspect of the disclosure relates to the protection of organoboronic compounds from degradation by C—B bond cleavage. The method comprises the aqueous hydrolysis of a boronic compound, e.g. boronic ester, for a period sufficiently short substantially to avoid cleavage of the C—B bond. By way of example, a period of no more than about 30 minutes at about room temperature may be mentioned.

Further included is the use of acetonitrile as a solvent in the preparation of organoboronate salts. In particular, an organoboronic acid is dissolved in acetonitrile and contacted with a base to form the corresponding organoboronate salt. A solid organoboronate salt containing water may be dried by azeodrying using acetonitrile.

Also provided is a process for separating diastereomers of a boronic acid of formula (IIa): 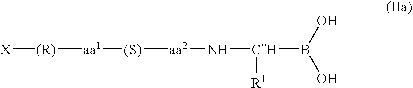
where:
X is H (to form NH₂) or an amino-protecting group;
aa¹ is an amino acid residue selected from Phe, Dpa and wholly or partially hydrogenated analogues thereof;
aa² is an imino acid residue having from 4 to 6 ring members;
R¹ is a group of the formula —(CH₂)ₛ-Z, where s is 2, 3 or 4 and Z is —OH, —OMe, —OEt or halogen (F, Cl, Br or I),

and where C* is a chiral centre,

the process comprising:

combining in diethylether solution (A) a boronic species selected from the boronic acid (I) and its esters, the boronic species including molecules having a chiral centre C* of (R) configuration and molecules having a chiral centre C* of (S) configuration, and (B) diethanolamine, the diethanolamine being in an amount of about 1.25±0.1 equivalents based on the boronic species in which the chiral centre C* is of (R) configuration, and mixing to form a mixture;
causing or allowing the boronic species and the diethanolamine to react until a precipitate forms; and
recovering the precipitate.

The precipitation step is selective for species having a chiral centre C* of (R) configuration, which are recovered in high purity.

The process may comprise converting the recovered precipitate to the acid of formula (I) by dissolving the precipitate in an organic solvent selected from halohydrocarbons and combinations thereof, agitating the resulting solution with an aqueous medium, for example an aqueous acid having a pH of below 3, whereby the dissolved precipitate is converted to the formula (I) acid, and recovering the formula (I) acid by evaporation.

One process of the disclosure comprises hydrolysing, e.g. allowing the hydrolysis of, a diethanolamine ester of an acid of formula (I) with an aqueous medium for a time sufficiently short for the product acid to be substantially free of impurity resulting from carbon-boron bond cleavage.

One class of processes further comprises converting the recovered acid of formula (I) to a pharmaceutically acceptable base addition salt thereof by dissolving the acid in acetonitrile, combining the resultant solution with an aqueous solution or suspension of a pharmaceutically acceptable base, and causing or allowing the base and the acid to react, then evaporating to dryness to obtain an evaporation residue.

The base addition salt may thereafter be incorporated in a pharmaceutical formulation.

The disclosure further includes a process for making a boronic acid of Formula (I) in which R¹ is of the formula —(CH₂)ₛ—O—R³ wherein R³ is methyl or ethyl and s is independently 2, 3 or 4, or for making a synthetic intermediate for such an acid, the process comprising:
reacting a 1-metalloalkoxyalkane, where the alkoxyalkane is of the formula —(CH₂)ₛ—O—R³, and a borate ester to form a compound of Formula (VI):
   (HO)₂B—(CH₂)ₛ—O—R³ (VI),
   the process optionally further comprising converting the compound of Formula (VI) into an acid of formula (I), for example by a known process.

In one class of processes, the compound of Formula (VI) is converted into an ester of the Formula (I) acid, which ester is transesterified with diethanolamine to form a precipitate. The precipitate may then be recovered for further processing. Suitably, the diethanolamine transesterification is used for resolving chiral isomers, as described herein. The resolved active R,S,R isomer may then be converted from the diethanolamine ester to the free acid, for example as described herein, and the free acid may if desired be converted to a salt, for example as described herein.

The disclosure includes the products of the aforesaid processes. Further products are described and claimed in the following specification.

The processes and products described herein may be performed or, as the case may be, provided on mass or commercial scale.

There is a debate in the literature as to whether boronates in aqueous solution form the ‘trigonal’ —B(OH)₂ or ‘tetrahedral’ —B(OH)₃⁻ boron species, but NMR evidence seems to indicate that at a pH below the first pKa of the boronic acid the main boron species is the neutral —B(OH)₂. In the duodenum the pH is likely to be between 6 and 7, so the trigonal species is likely to be predominant here. In any event, the symbol —B(OH)₂ includes tetrahedral as well as trigonal boron species, and throughout this specification symbols indicating trigonal boron species embrace also tetrahedral species.

The present disclosure is not limited as to the exact identity of the boronic/boronate moieties in the salts, provided that they contain a boronate species derived from a boronic acid (e.g. of formula (I)) and a counter-ion. Such boronate species may be boronate anions in any equilibrium form thereof. Boronates in the solid phase may form anhydrides and the disclosed boronate salts when in the solid phase may comprise boronate anhydrides, as a boronic equilibrium species. The disclosure therefore encompasses pharmaceutical formulations containing a boronate species as active principle and a counter-ion.

Further aspects and embodiments of the disclosure are set forth in the following description and claims.

Throughout the description and claims of this specification, the words “comprise” and “contain” and variations of the words, for example “comprising” and “comprises”, mean “including but not limited to”, and are not intended to (and do not) exclude other moieties, additives, components, integers or steps.

### DETAILED DESCRIPTION OF SEVERAL EXAMPLES

Glossary

The following terms and abbreviations are used in this specification:

The expression “acid salt” as applied to a salt of a boronic acid refers to salts of which a single —OH group of the trigonally-represented acid group —B(OH)₂ is deprotonated. Thus salts wherein the boronate group carries a single negative charge and may be represented as —B(OH)(O⁻) or as [—B(OH)₃]⁻ are acid salts. The expression encompasses salts having a cation having a valency n wherein the molar ratio of boronic acid to cation is approximately n to 1. In practical terms, the observed stoichiometry is unlikely to be exactly n:1 but will be consistent with a notional n:1 stoichiometry. For example, the observed mass of the cation might vary from the calculated mass for a n:1 stoichiometry by no more than about 10%, e.g. no more than about 7.5%; in some cases an observed mass of a cation might vary from the calculated mass by no more than about 1%. Calculated masses are suitably based on the trigonal form of the boronate. (At an atomic level, a salt stoichiometrically consistent with being an acid salt might contain boronates in a mix of protonation states, whose average approximates to single deprotonation and such “mixed” salts are included in the term “acid salt”).

α-Aminoboronic acid or Boro(aa) refers to an amino acid in which the CO₂ group has been replaced by BO₂.

The term “amino-group protecting moiety” refers to any group used to derivatise an amino group, especially an N-terminal amino group of a peptide or amino acid. Such groups include, without limitation, alkyl, acyl, alkoxycarbonyl, aminocarbonyl, and sulfonyl moieties. However, the term “amino-group protecting moiety” is not intended to be limited to those particular protecting groups that are commonly employed in organic synthesis, nor is it intended to be limited to groups that are readily cleavable.

The term “equilibrium form” refers to differing forms of the same compounds which may be represented in an equilibrium equation, as in the case of a boronic acid in equilibrium with a boronic anhydride and/or in equilibrium with one or more different boronate ions or as in the case of an organic base in equilibrium with a protonated form thereof.

The phrase “pharmaceutically acceptable” is employed herein to refer to those compounds, materials, compositions, and/or dosage forms which are, within the scope of sound medical judgment, suitable for use in contact with the tissues of human beings or animals without excessive toxicity, irritation, allergic response, or other problem or complication, commensurate with a reasonable benefit/risk ratio.

The expression “thrombin inhibitor” refers to a product which, within the scope of sound pharmacological judgement, is potentially or actually pharmaceutically useful as an inhibitor of thrombin, and includes reference to substance which comprises a pharmaceutically active species and is described, promoted or authorised as a thrombin inhibitor. Such thrombin inhibitors may be selective, that is they are regarded, within the scope of sound pharmacological judgement, as selective towards thrombin in contrast to other proteases; the term “selective thrombin inhibitor” includes reference to substance which comprises a pharmaceutically active species and is described, promoted or authorised as a selective thrombin inhibitor.

The term “heteroaryl” refers to a ring system which has at least one (e.g. 1, 2 or 3) in-ring heteroatoms and has a conjugated in-ring double bond system. The term “heteroatom” includes oxygen, sulfur and nitrogen, of which sulfur is sometimes less preferred.

“Natural amino acid” means an L-amino acid (or residue thereof) selected from the following group of neutral (hydrophobic or polar), positively charged and negatively charged amino acids:

Hydrophobic Amino Acids
A=Ala=alanine
V=Val=valine
I=Ile=isoleucine
L=Leu=leucine
M=Met=methionine
F=Phe=phenylalanine
P=Pro=proline
W=Trp=tryptophan

Polar (Neutral or Uncharged) Amino Acids
N=Asn=asparagine
C=Cys=cysteine
Q=Gln=glutamine
G=Gly=glycine
S=Ser=serine
T=Thr=threonine
Y=Tyr=tyrosine

Positively Charged (Basic) Amino Acids
R=Arg=arginine
H=His=histidine
K=Lys=lysine

Negatively Charged Amino Acids
D=Asp=aspartic acid
E=Glu=glutamic acid.
ACN=acetonitrile
Amino acid=α-amino acid
Base addition salt=a salt which is prepared from addition of an inorganic base or an organic base to a free acid (in this case the boronic acid)
Cbz=benzyloxycarbonyl
Cha=cyclohexylalanine (a hydrophobic unnatural amino acid)
Charged (as applied to drugs or fragments of drug molecules, e.g. amino acid residues)=carrying a charge at physiological pH, as in the case of an amino, amidino or carboxy group
Dcha=dicyclohexylalanine (a hydrophobic unnatural amino acid)
Dpa=diphenylalanine (a hydrophobic unnatural amino acid)
Drug=a pharmaceutically useful substance, whether the active in vivo principle or a prodrug
Mpg=3-methoxypropylglycine (a hydrophobic unnatural amino acid)
Multivalent=valency of at least two, for example two or three
Neutral (as applied to drugs or fragments of drug molecules, e.g. amino acid residues)=uncharged=not carrying a charge at physiological pH
Pinac=Pinacol=2,3-dimethyl-2,3-butanediol
Pinanediol=2,3-pinanediol=2,6,6-trimethylbicyclo[3.1.1]heptane-2,3-diol
Pip=pipecolinic acid
Room temperature=25° C.±2° C.
RT=retention time
Strong base=a base having a sufficiently high pKb to react with a boronic acid. Suitably such bases have a pKb of 7 or more, e.g. 7.5 or more, for example about 8 or more
THF=tetrahydrofuran
Thr=thrombin

The following description commences with a discussion of the types of compounds to which the described processes may be applied. There are then described new findings relating to purity and stability. The processes are described next, and then the specific products of the processes.

The Compounds

The disclosure relates to organoboronic compounds (organoboronates) and particularly aminoboronates (aminoboronic compounds) and compounds comprising peptide boronate (boronic) moieties. The compounds may be boronic acids which have a neutral aminoboronic acid residue capable of binding to the thrombin S1 subsite linked through a peptide linkage to a hydrophobic moiety capable of binding to the thrombin S2 and S3 subsites. As examples may be mentioned compounds of the formula YCO—NHC*(R²)H—B(OH)₂ (formula I) where Y comprises a moiety which, together with R², has affinity for the substrate binding site of thrombin and R² is a hydrophobic moiety having affinity for the thrombin S1 subsite. R² may be a moiety R⁹ which is a straight chain alkyl group interrupted by one or more ether linkages and in which the total number of oxygen and carbon atoms is from 3 to 6, or is —(CH₂)ₘ—W where m is from 2 to 5 and W is —OH or halogen (F, Cl, Br or I). C* is a chiral centre. As examples of straight chain alkyl interrupted by one or more ether linkages (—O—) may be mentioned alkoxyalkyl (one interruption) and alkoxyalkoxyalkyl (two interruptions). R⁹ is an alkoxyalkyl group in one subset of compounds, e.g. alkoxyalkyl containing 4 carbon atoms.

Typically, YCO— comprises an amino acid residue (whether natural or unnatural) which binds to the S2 subsite of thrombin, the amino acid residue being N-terminally linked to a moiety which binds the S3 subsite of thrombin.

In one class of Formula (I) acids, YCO— is an optionally N-terminally protected dipeptide residue which binds to the S3 and S2 binding sites of thrombin and the peptide linkages in the acid are optionally and independently N-substituted by a C₁-C₁₃ hydrocarbyl group optionally containing in-chain and/or in-ring nitrogen, oxygen or sulfur and optionally substituted by a substituent selected from halo, hydroxy and trifluoromethyl. The N-terminal protecting group, when present, may be a group X as defined above (other than hydrogen). Normally, the acid contains no N-substituted peptide linkages; where there is an N-substituted peptide linkage, the substituent is often 1C to 6C hydrocarbyl, e.g. saturated hydrocarbyl; the N-substituent comprises a ring in some embodiments, e.g. cycloalkyl, and may be cyclopentyl, for example. One class of acids has an N-terminal protecting group (e.g. an X group) and unsubstituted peptide linkages.

Where YCO— is a dipeptide residue (whether or not N-terminally protected), the S3-binding amino acid residue may be of R configuration and/or the S2-binding residue may of S configuration. The fragment —NHCH(R⁹)—B(OH) may of R configuration. The disclosure is not restricted to chiral centres of these conformations, however.

In one class of compounds, the side chain of P3 (S3-binding) amino acid and/or the P2 (S2-binding) amino acid is a moiety other than hydrogen selected from a group of formula A or B:
—(CO)ₐ—(CH₂)_{b}-D_{c}-(CH₂)_{d}-E (A)
—(CO)ₐ—(CH₂)_{b}-D_{c}-Cₑ(E¹)(E²)(E³) (B)
wherein
a is 0 or 1;
e is 1;
b and d are independently 0 or an integer such that (b+d) is from 0 to 4 or, as the case may be, (b+e) is from 1 to 4;
c is 0 or 1;
D is O or S;
E is H, C₁-C₆ alkyl, or a saturated or unsaturated cyclic group which normally contains up to 14 members and particularly is a 5-6 membered ring (e.g. phenyl) or an 8-14 membered fused ring system (e.g. naphthyl), which alkyl or cyclic group is optionally substituted by up to 3 groups (e.g. 1 group) independently selected from C₁-C₆ trialkylsilyl, —CN, —R¹³, —R¹²OR¹³, —R¹²COR¹³, —R¹²CO₂R¹³ and —R¹²O₂CR¹³, wherein R¹² is —(CH₂)_{f}— and R¹³ is —(CH₂)_{g}H or by a moiety whose non-hydrogen atoms consist of carbon atoms and in-ring heteroatoms and number from 5 to 14 and which contains a ring system (e.g. an aryl group) and optionally an alkyl and/or alkylene group, wherein f and g are each independently from 0 to 10, g particularly being at least 1 (although —OH may also be mentioned as a substituent), provided that (f+g) does not exceed 10, more particularly does not exceed 6 and most particularly is 1, 2, 3 or 4, and provided that there is only a single substituent if the substituent is a said moiety containing a ring system, or E is C₁-C₆ trialkylsilyl; and E¹, E² and E³ are each independently selected from —R¹⁵ and -J-R¹⁵, where J is a 5-6 membered ring and R¹⁵ is selected from C₁-C₆ trialkylsilyl, —CN, —R¹³, —R¹²OR¹³, —R¹²COR¹³, —R¹²CO₂R¹³, —R¹²O₂CR¹³, and one or two halogens (e.g. in the latter case to form a -J-R¹⁵ moiety which is dichlorophenyl), where R¹² and R¹³ are, respectively, an R¹² moiety and an R¹³ moiety as defined above (in some acids where E¹, E² and E³ contain an R¹³ group, g is 0 or 1);
in which moiety of Formula (A) or (B) any ring is carbocyclic or aromatic, or both, and any one or more hydrogen atoms bonded to a carbon atom is optionally replaced by halogen, especially F.

In certain examples, a is 0. If a is 1, c may be 0. In particular examples, (a+b+c+d) and (a+b+c+e) are no more than 4 and are more especially 1, 2 or 3. (a+b+c+d) may be 0.

Exemplary groups for E, E¹, E² and E³ include aromatic rings such as phenyl, naphthyl, pyridyl, quinolinyl and furanyl, for example; non-aromatic unsaturated rings, for example cyclohexenyl; saturated rings such as cyclohexyl, for example. E may be a fused ring system containing both aromatic and non-aromatic rings, for example fluorenyl. One class of E, E¹, E² and E³ groups are aromatic (including heteroaromatic) rings, especially 6-membered aromatic rings. In some compounds, E¹ is H whilst E² and E³ are not H; in those compounds, examples of E² and E³ groups are phenyl (substituted or unsubstituted) and C₁-C₄ alkyl, e.g. methyl.

In one class of embodiments, E contains a substituent which is C₁-C₆ alkyl, (C₁-C₅ alkyl)carbonyl, carboxy C₁-C₅ alkyl, aryl (including heteroaryl), especially 5-membered or preferably 6-membered aryl (e.g. phenyl or pyridyl), or arylalkyl (e.g. arylmethyl or arylethyl where aryl may be heterocyclic and is preferably 6-membered).

In another class of embodiments, E contains a substituent which is OR¹³, wherein R¹³ can be a 6-membered ring, which may be aromatic (e.g. phenyl) or is alkyl (e.g. methyl or ethyl) substituted by such a 6-membered ring.

A class of moieties of formula A or B are those in which E is a 6-membered aromatic ring optionally substituted, particularly at the 2-position or 4-position, by —R¹³ or —OR¹³.

The disclosure includes salts in which the P3 and/or P2 side chain comprises a cyclic group in which 1 or 2 hydrogens have been replaced by halogen, e.g. F or Cl.

The disclosure includes a class of salts in which the side chains of formula (A) or (B) are of the following formulae (C), (D) or (E): 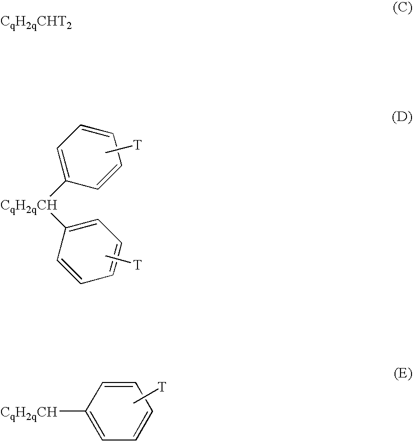
wherein q is from 0 to 5, e.g. is 0, 1 or 2, and each T is independently hydrogen, one or two halogens (e.g. F or Cl), —SiMe₃, —CN, —R¹³, —OR¹³, —COR¹³, —CO₂R¹³ or —O₂CR¹³. In some embodiments of structures (D) and (E), T is at the 4-position of the phenyl group(s) and is —R¹³, —OR¹³, —COR¹³, —CO₂R¹³ or —O₂CR¹³, and R¹³ is C₁-C₁₀ alkyl and more particularly C₁-C₆ alkyl. In one sub-class, T is —R¹³ or —OR¹³, for example in which f and g are each independently 0, 1, 2 or 3; in some side chains groups of this sub-class, T is —R¹²OR¹³ and R¹³ is H.

In one class of the moieties, the side chain is of formula (C) and each T is independently R¹³ or OR¹³ and R¹³ is C₁-C₄ alkyl. In some of these compounds, R¹³ is branched alkyl and in others it is straight chain. In some moieties, the number of carbon atoms is from 1 to 4.

In many dipeptide fragments YCO— (which dipeptides may be N-terminally protected or not), the P3 amino acid has a side chain of formula (A) or (B) as described above and the P2 residue is of an imino acid.

The disclosure relates in particular to medicaments comprising salts, e.g. metal salts, of organoboronic acids which are thrombin inhibitors, particularly selective thrombin inhibitors, having a neutral P1 (S1-binding) moiety. For more information about moieties which bind to the S3, S2 and S1 sites of thrombin, see for example Tapparelli C et al, Trends Pharmacol. Sci. 14: 366-376, 1993; Sanderson P et al, Current Medicinal Chemistry, 5: 289-304, 1998; Rewinkel J et al, Current Pharmaceutical Design, 5:1043-1075, 1999; and Coburn C Exp. Opin. Ther. Patents 11(5): 721-738, 2001. The thrombin inhibitory organoboronic acids to which this specification relates are not limited to those having S3, S2 and S1 affinity groups described in the publications listed in the preceding sentence.

The boronic acids may have a Ki for thrombin of about 100 nM or less, e.g. about 20 nM or less.

An exemplary acid is TRI 50c, whose tripeptide sequence has three chiral centres. The Phe residue is considered to be of (R)-configuration and the Pro residue of natural (S)-configuration, at least in compounds with commercially useful inhibitor activity; the Mpg residue is believed to be of (R)-configuration in isomers with commercially useful inhibitor activity. Thus, the active, or most active, TRI 50c stereoisomer is considered to be of (R,S,R)-configuration and may be represented as:

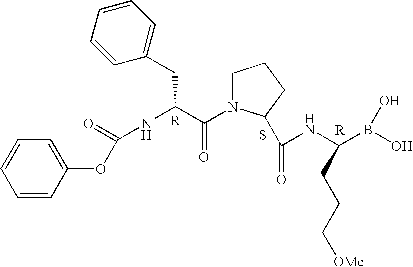

### Cbz-(R)-Phe-(S)-Pro-(R)-boroMpg-OH

### (R,S,R)-TRI 50c

In other compounds of the formula YCO—NHC*(R²)H—B(OH)₂ where YCO is an optionally N-terminally protected dipeptide residue, the active or most active isomers similarly have P3 and P2 residues respectively of (R)-and (S)-configuration and a chiral centre C* of (R)-configuration.

The disclosure relates also to a narrower class of boronic acids which includes TRI 50c, viz acids of formula (II): 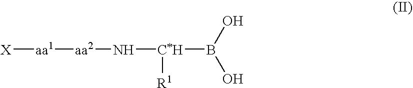
where:
X is H (to form NH₂) or an amino-protecting group;
aa¹ is an amino acid selected from Phe, Dpa and wholly or partially hydrogenated analogues thereof, the wholly hydrogenated analogues being Cha and D-Dcha;
aa² is an imino acid having from 4 to 6 ring members;
R¹ is a group of the formula —(CH₂)ₛ-Z, where s is 2, 3 or 4 and Z is —OH, —OMe, —OEt or halogen (F, Cl, Br or I),
and where C* is a chiral centre.

Also to be mentioned is a class of compounds corresponding to those of formula (II) but in which R¹ is replaced by R⁹ as defined previously.

In one class of compounds, X is R⁶—(CH₂)ₚ—C(O)—, R⁶—(CH₂)ₚ—S(O)₂—, R⁶—(CH₂)ₚ—NH—C(O)— or R⁶—(CH₂)ₚ—O—C(O)— wherein p is 0, 1, 2, 3, 4, 5 or 6 (of which 0 and 1 are preferred and R⁶ is H or a 5 to 13-membered cyclic group optionally substituted by 1, 2 or 3 substituents selected from halogen, amino, nitro, hydroxy, a C₅-C₆ cyclic group, C₁-C₄ alkyl and C₁-C₄ alkyl containing, and/or linked to the 5 to 13-membered cyclic group through, an in-chain O, the aforesaid alkyl groups optionally being substituted by a substituent selected from halogen, amino, nitro, hydroxy and a C₅-C₆ cyclic group. More preferably X is R⁶-(CH₂)ₚ—C(O)— or R⁶—(CH₂)ₚ—O—C(O)— and p is 0 or 1. Said 5 to 13-membered cyclic group is often aromatic or heteroaromatic, for example is a 6-membered aromatic or heteroaromatic group. In many cases, the group is not substituted.

Exemplary X groups are (2-pyrazine) carbonyl, (2-pyrazine) sulfonyl and particularly benzyloxycarbonyl.

A particular class of acids comprises those in which aa² is a residue of an imino acid of formula (IV): 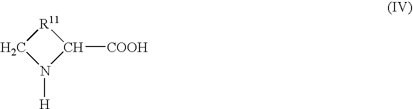
where R¹¹ is —CH₂—, CH₂—CH₂—, —S—CH₂— or —CH₂—CH₂—CH₂—, which group when the ring is 5 or 6-membered is optionally substituted at one or more —CH₂— groups by from 1 to 3 C₁-C₃ alkyl groups, for example to form the R¹¹ group —S—C(CH₃)₂—. Of these imino acids, azetidine-2-carboxylic acid, especially (s)-azetidine-2-carboxylic acid, and more particularly proline are preferred.

It will be appreciated from the above that a very preferred class of products consists of those in which aa¹-aa² is Phe-Pro. In another preferred class, aa¹-aa² is Dpa-Pro. In other products, aa¹-aa² is Cha-Pro or Dcha-Pro. Of course, the disclosure includes corresponding product classes in which Pro is replaced by (s)-azetidine-2-carboxylic acid.

As already indicated, R¹ is a moiety of the formula —(CH₂)ₛ-Z. Integer s is 2, 3 or 4 and W is —OH, —OMe, —OEt or halogen (F, Cl, I or, preferably, Br). The most preferred Z groups are —OMe and —OEt, especially —OMe. It is preferred that s is 3 for all Z groups and, indeed, for all compounds of the disclosure. Particularly preferred R¹ groups are 2-bromoethyl, 2-chloroethyl, 2-methoxyethyl, 4-bromobutyl, 4-chlorobutyl, 4-methoxybutyl and, especially, 3-bromopropyl, 3-chloropropyl and 3-methoxypropyl. Most preferably, R¹ is 3-methoxypropyl. 2-Ethoxyethyl is another preferred R¹ group.

In the case of thrombin inhibitors of formula (I), the active or most active stereoisomer has an aa¹ moiety of (R) configuration, an aa² moiety of (S) configuration and a chiral centre C* of (R) configuration.

Accordingly, particular acids are of formula III:
X—(R)-Phe-(S)-Pro-(R)-Mpg-B(OH)₂ III,
especially Cbz-(R)-Phe-(S)-Pro-(R)-Mpg-B(OH)₂ (i.e. (R,S,R) TRI 50c); also to be mentioned are analogues of these compounds in which Mpg is replaced by a residue with another of the particularly preferred R¹ groups and/or Phe is replaced by Dpa or another aa¹ residue.

As suitable salts may be mentioned salts of metals, e.g. of monovalent or divalent metals, and stronger organic bases, for example:
1. Alkali metal salts;
2. Divalent, e.g. alkaline earth metal, salts;
3. Group III metals;
4. Salts of strongly basic organic nitrogen-containing compounds, including:
   4A. Salts of guanidines and their analogues;
   4B. Salts of strongly basic amine, examples of which include (i) aminosugars and (ii) other amines.

Of the above salts, particularly illustrative are alkali metals, especially Na and Li. Also illustrative are aminosugars.

Specific salts are of the acid boronate though in practice the acid salts may contain a very small proportion of the doubly deprotonated boronate. The term “acid boronate” refers to trigonal —B(OH)₂ groups in which one of the B—OH groups is deprotonated as well as to corresponding tetrahedral groups in equilibrium therewith. Acid boronates have a stoichiometry consistent with single deprotonation.

Disregarding chirality considerations for the moment, the disclosure includes therefore products (compositions of matter) which comprise salts which may be represented by formula (V): 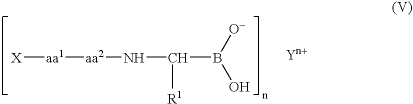
where Yⁿ⁺ is a pharmaceutically acceptable cation obtainable from a strong base, and aa¹, aa², X and R¹ are as defined above. Also included are products in which R¹ is replaced by another R⁹ group.

One class of salts have a solubility of about 10 mM or more, e.g. of at least about 20 mM, when their solubility is determined as described in the examples at a dissolution of 25 mg/ml. More particularly yet they have a solubility of least 50 mM when their solubility is determined as described in the examples at a dissolution of 50 mg/ml.

The disclosure includes products which comprise salts of boronic acids (I) having an observed stoichiometry consistent with the salt being of (being representable by) the formula “(boronate⁻)ₙ cationⁿ⁺”. One class of such salts are represented by the formula:
[Cbz-(R)-Phe-(S)-Pro-(R)-Mpg-B(OH)(O⁻)]M⁺
where M⁺ represents a monovalent cation, especially an alkali metal cation. It will be understood that the above representation is a notional representation of a product whose observed stoichiometry is unlikely to be literally and exactly 1:1. In any event, a particular salt is Cbz-(R)-Phe-(S)-Pro-(R)-Mpg-B(OH)₂ monosodium salt (TGN 255). In the above formula, the trigonally-represented boronate represents, as always, boronates which are trigonal, tetrahedral or mixed trigonal/tetrahedral.

Particularly exemplary are products which comprise:
(i) species selected from (a) acids of formula (VIII): X—(R)-Phe-(S)-Pro-(R)-Mpg-B(OH)₂ where X is H or an amino-protecting group, especially Cbz, (b) boronate anions thereof, and (c) any equilibrium form of the aforegoing (e.g. an anhydride); and
(ii) ions having a valency n in combination with said species, the species and said ions having an observed stoichiometry consistent with a notional species:ion stoichiometry of n:1. In one class of salts, n is 1.

The counter-ions for the boronate ions described herein are considered in turn below with reference to formulae (VI), (VII), (VIII), (IX) and (X). These formulae again do not take account of chirality.

1. Monovalent Metal, Especially Alkali Metal Salts

Suitable alkali metals include lithium, sodium and potassium. All of these are remarkably soluble. Lithium and sodium are illustrative because of their high solubility. The lithium and particularly sodium salts are of surprisingly high solubility in relation to potassium amongst others. Sodium is most used in many instances. Salts containing mixtures of alkali metals are contemplated by the disclosure.

The disclosure includes products comprising salts of the formula (VI) 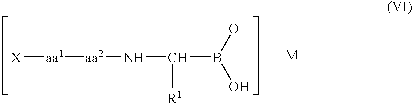
where M⁺ is an alkali metal ion and aa¹ , aa², X and R¹ are as defined above, as well as salts in which both hydroxy groups of the boronate group are in salt form (preferably with another identical M⁺ group) and mixtures of such salts. Included also are products wherein R¹ is replaced by another R⁹ group.

In some embodiments, alkali metal salts, notably sodium, are incorporated in parenteral, e.g. intravenous, formulations.

2. Divalent. e.g. Alkaline Earth Metal (Group II Metal) Salts

One example of a divalent metal is calcium. Another suitable divalent metal is magnesium. Also contemplated is zinc. The divalent metals are usually used in a boronic acid:metal ratio of substantially 2:1, in order to achieve the preferred monovalent boronate moiety. Salts containing mixtures of divalent metals, e.g. mixtures of alkaline earth metals, are also contemplated.

Further disclosed are products (compositions of matter) which comprise salts which may be represented by the formula (VII): 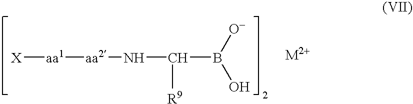
where M²⁺ is a divalent metal cation, e.g. an alkaline earth metal or zinc cation, and aa¹, aa², X and R⁹ are as defined above, as well as salts in which both hydroxy groups of the boronate group are deprotonated and mixtures of such salts. As previously indicated, the boronate may comprise a tetrahedral species.

In some embodiments, multivalent metal salts, e.g. calcium or magnesium salts or other divalent metal salts, are incorporated in oral formulations.

3. Group III Metals

Suitable Group III metals include aluminium and gallium. Salts containing mixtures of Group III metals are also contemplated.

The disclosure includes products comprising salts of the formula (VIII): 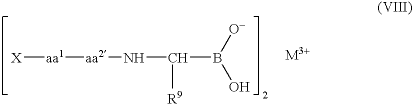
where M³⁺ is a Group III metal ion and aa¹, aa^{2′}, X and R⁹ are as defined above, as well as salts in which both hydroxy groups of the boronate group are in salt form and mixtures of such salts. As previously indicated, the boronate may comprise a tetrahedral species.
4. Strongly Basic Organic Nitrogen-Containing Compounds

The disclosure includes products obtainable by (having the characteristics of a product obtained by) reaction of a peptide boronic acid as defined above and a strong organic base. Two illustrative classes of organic base are described in sections 4A and 4B below. Particularly preferred are acid salts (in which one of the two boronic —OH groups is deprotonated). Most commonly, the salts contain a single type of organic counter-ion (disregarding trace contaminants) but the disclosure contemplates salts containing mixtures of organic counter-ions; in one sub-class, the different counter-ions all fall within the section 4A family described below or, as the case may be, in the section 2B family below; in another subclass, the salts comprise a mixture of organic counter-ions which are not all from the same family (4A or 4B).

Suitable organic bases include those with a pKb of 7 or more, e.g. 7.5 or more, for example in the region of 8 or more. Bases which are less lipophilic [e.g. have at least one polar functional group (e.g. 1, 2 or 3 such groups) for example hydroxy] are favoured; thus aminosugars are one favoured class of base.

4A. Guanidines and Their Analogues

The guanidino compound (guanidine) may in principle be any soluble and pharmaceutically acceptable compound having a guanidino or a substituted guanidino group, or a substituted or unsubstituted guanidine analogue. Suitable substituents include aryl (e.g. phenyl), alkyl or alkyl interrupted by an ether or thioether linkage and, in any event, typically contain from 1 to 6 and especially 1, 2, 3, or 4 carbon atoms, as in the case of methyl or ethyl. The guanidino group may have 1, 2, 3 or 4 substituent groups but more usually has 1 or 2 substituent groups, for instance on a terminal nitrogen. One class of guanidines is monoalkylated; another class is dialkylated. As guanidine analogues may be mentioned thioguanidines and 2-amino pyridines. Compounds having unsubstituted guanidino groups, for example guanidine and arginine, form one particular class.

Salts containing mixtures of guanidines are contemplated by the disclosure.

A particular guanidino compound is L-arginine or an L-arginine analogue, for example D-arginine, or the D- or, preferably, L-isomers of homoarginine or agmatine [(4-aminobutyl) guanidine]. Less preferred arginine analogues are NG-nitro-L-arginine methyl ester, for example, and constrained guanidine analogues, particularly 2-amino pyrimidines, for example 2,6-quinazolinediamines such as 5,6,7,8-tetrahydro-2,6-quinazolinediamine, for example. The guanidino compound may also be a peptide, for example a dipeptide, containing arginine; one such dipeptide is L-tyrosyl-L-arginine.

Some particular guanidino compounds are compounds of formula (XXX): 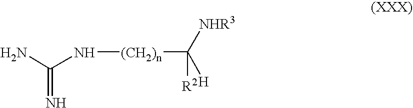
where n is from 1 to 6 and for example at least 2, e.g. 3 or more, and in many instances no more than 5. Most particularly, n is 3, 4 or 5. R² is H or carboxylate or derivatised carboxylate, for example to form an ester (e.g. a C₁-C₄ alkyl ester) or amide. R³ is H, C₁-C₄ alkyl or a residue of a natural or unnatural amino acid (e.g. tyrosine). The compounds of formula (IV) are usually of L-configuration. The compounds of formula (IV) are arginine (n=3; R²=carboxyl; R³═H) and arginine derivatives or analogues.

The disclosure includes products comprising salts of the formula (IX) 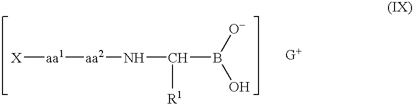
where aa¹, aa², X and R¹ are as defined previously and G⁺ is the protonated form of a pharmaceutically acceptable organic compound comprising a guanidino group or an analogue thereof, as well as salts in which both hydroxy groups of the boronate group are in salt form (preferably with another identical G⁺ group) and mixtures of such salts. Also included are products wherein R¹ is replaced by another R⁹ group.
4B8 Strongly Basic Amines

The disclosure includes products obtainable by (having the characteristics of a product obtained by) reaction of a peptide boronic acid as defined above and a strong organic base which is an amine. The amine may in principle be any soluble and pharmaceutically acceptable amine.

It is envisaged that a desirable class of amine includes those having polar functional groups in addition to a single amine group, as such compounds will be more hydrophilic and thus more soluble than others. In certain salts, the or each additional functional group is hydroxy. Some amines have 1, 2, 3, 4, 5 or 6 additional functional groups, especially hydroxy groups. In one illustrative class of amines the ratio of (amino plus hydroxy groups): carbon atoms is from 1:2 to 1:1, the latter ratio being particularly preferred. These amines with one or more additional polar functional groups may be a hydrocarbon, especially an alkane, substituted by the amino group and the additional polar group(s). The amino group may be substituted or unsubstituted and, excluding amino substituents, the polar base may contain, for example, up to 10 carbon atoms; usually there are no less than three such carbon atoms, e.g. 4, 5 or 6. Aminosugars are included in this category of polar bases.

The disclosure includes products comprising salts of the formula (X) 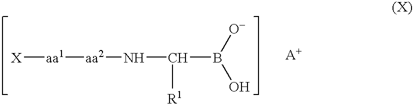
where aa¹, aa², X and R¹ are as defined previously and A⁺ is the protonated form of a pharmaceutically acceptable amine, as well as salts in which both hydroxy groups of the boronate group are in salt form (preferably with another identical A⁺ group) and mixtures of such salts. In one class of such products, A⁺ is the protonated form of an amine described in section 2B(i) below; in another class A⁺ is the protonated form of an amine described in 2B(ii) below. Also included are products in which R¹ is replaced by another R⁹ group. Two illustrative classes of amine base are described in sections 4B(i) and 4B(ii) below. Particularly preferred are acid salts (in which one of the two boronic —OH groups is deprotonated). Most commonly, the salts contain a single type of amine counter-ion (disregarding trace contaminants) but the disclosure contemplates salts containing mixtures of amine counter-ions; in one sub-class, the different counter-ions all fall within the sub-section 4B(i) family described below or, as the case may be, in the sub-section 4B(ii) family below; in another subclass, the salts comprise a mixture of organic counter-ions which are not all from the same family (4B(i) or 4B(ii)).

4B(i) Aminosugars

The identity of the aminosugar is not critical. Preferred aminosugars include ring-opened sugars, especially glucamines. Cyclic aminosugars are also envisaged as useful. One class of the aminosugars is N-unsubstituted and another, preferred, class is N-substituted by one or two N-substituents (e.g. one). Suitable substituents are hydrocarbyl groups, for example and without limitation containing from 1 to 12 carbon atoms; the substituents may comprise alkyl or aryl moieties or both. Exemplary substituents are C₁, C₂, C₃, C₄, C₅, C₆, C₇ and C₈ alkyl groups, in particular methyl and ethyl, of which methyl is illustrative. Data indicate that aminosugars, especially N-methyl-D-glucamine, are of surprisingly high solubility.

A most preferred aminosugar is N-methyl-D-glucamine:

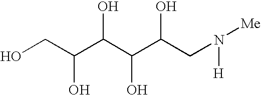

4B(ii) Other Amines

Other suitable amines include amino acids (whether naturally occurring or not) whose side chain is substituted by an amino group, especially lysine.

Some amines are compounds of formula (XI): 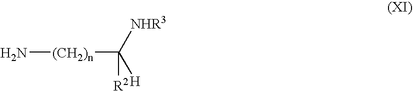
where n, R² and R³ are as defined in relation to formula (IV). The compounds of formula (VI) are usually of L-configuration. The compounds of formula (VI) are lysine (n=4; R²=carboxyl; R³═H) and lysine derivatives or analogues. A most preferred amine is L-lysine.

Other suitable amines are nitrogen-containing heterocycles. At least usually, such heterocyclic compounds are alicyclic; one class of the heterocyclic compounds is N-substituted and another, preferred, class is N-unsubstituted. The heterocycles may contain 6 ring-forming atoms, as in the cases of piperidine, piperazine and morpholine. One class of amines includes N-containing heterocycles substituted by polar substituents, especially hydroxy, e.g. 1, 2 or 3 times.

The disclosure therefore includes amines other than aminosugars which have one or more (e.g. 1, 2, 3, 4, 5 or 6) polar substituents, especially hydroxy, in addition to one amine group. Such compounds may have a ratio of (amino plus hydroxy groups):carbon atoms of 1:2 to 1:1, the latter ratio being particularly preferred.

The disclosure includes mixed salts, i.e. salts containing a mixture of boropeptide moieties and/or counterions but single salts are preferred.

In one class, the salts have a solubility of at least 10 mM, e.g. at least 20 mM, when their solubility is determined as described in the examples at a dissolution of 25 mg/ml. In another class they have a solubility of least 50 mM when their solubility is determined as described in the examples at a dissolution of 50 mg/ml.

Further to be mentioned are salts wherein the counter-ion to the boronic acid is not ammonium or choline.

There are included salts of boronic acids (I) having an observed stoichiometry consistent with the salt being of (being representable by) the formula “(boronate⁻)ₙ cationⁿ⁺”. Still disregarding chirality, one class of such salts are represented by the formula:
[Cbz-Phe-Pro-Mpg-B(OH)(O⁻)]M⁺
where M⁺ represents a monovalent cation, especially an alkali metal cation. It will be understood that the above representation is a notional representation of a product whose observed stoichiometry is unlikely to be literally and exactly 1:1. In the above formula, the trigonally-represented boronate represents, as always, boronates which are trigonal, tetrahedral or mixed trigonal/tetrahedral.

Another class of such salts are represented by the formula:
[Cbz-Phe-Pro-Mpg-B(OH)(O⁻)]₂M²⁺
where M²⁺ represents a divalent cation, especially an alkaline earth metal cation. It will be understood that the above representation is a notional representation of a product whose observed stoichiometry is unlikely to be literally and exactly 2:1.

The disclosure additionally includes lithium and sodium salts of boronic acid drugs having an observed stoichiometry consistent with the salt being of (being representable by) the formula “(boronate⁻) Na⁺” or “(boronate⁻) Li⁺”. In other salts of this type, the metal is potassium. One class of salts having such stoichiometry comprises salts of boronic acids of formula (III), as for example in the case of a salt of the formula:
[Cbz-Phe-Pro-Mpg-B(OH)(O⁻)]Na⁺.

The disclosure includes salts of the above formula in which Na⁺ is replaced by Li⁺. Also included are corresponding potassium salts. It will be understood that the above representation is a notional representation of a product whose observed stoichiometry is unlikely to be literally and exactly 1:1. In the above formula, the trigonally-represented boronate represents boronates which are trigonal, tetrahedral or mixed trigonal/tetrahedral.

The disclosure additionally includes calcium and magnesium salts of boronic acid drugs having an observed stoichiometry consistent with the salt being of (being representable by) the formula “(boronate⁻)₂ Ca²⁺” or “(boronate⁻)₂ Mg²⁺”. In other salts of this type, the metal is zinc. One class of salts having such stoichiometry comprises salts of boronic acids of formula (III), as for example in the case of salts of the formula:
[Cbz-Phe-Pro-Mpg-B(OH)(O⁻)]₂ Ca²⁺.

The disclosure includes salts of the above formula in which Ca²⁺ is replaced by Mg²⁺. Also included are corresponding zinc salts. It will be understood that the above representation is a notional representation of a product whose observed stoichiometry is unlikely to be literally and exactly 2:1. In the above formula, the trigonally-represented boronate represents boronates which are trigonal, tetrahedral or mixed trigonal/tetrahedral.

Particularly exemplary are products which comprise:
(i) species selected from (a) acids of formula X—(R)-Phe-(S)-Pro-(R)-Mpg-B(OH)₂ where X is H or an amino-protecting group, especially Cbz, (b) boronate anions thereof, and (c) any equilibrium form of the aforegoing (e.g. an anhydride); and
(ii) ions having a valency n in combination with said species, the species and said ions having an observed stoichiometry consistent with a notional species:ion stoichiometry of n:1. In one class of salts, n is 1.
   Stability and Purity of the Compounds

Existing publications teach that organoboronic acids are degraded by oxidation of the C—B bond. See for example Wu et al (see above). Earlier work on the salts of TRI 50c confirmed that these salts and/or intermediates in their preparation are slightly unstable, to the extent that the salts were found to contain a boron-free impurity, designated impurity I, which was evidently generated by C—B bond cleavage. The salts are significantly more stable to such degradation than the free acid. (However, in single experiments, the ammonium salt of TRI 50c appeared to decompose on drying to yield ammonia, whilst the choline salt demonstrated rapid decomposition to Impurity I. Although experiments have not been conducted to reproduce these unrepeated observations, there is provided a sub-class in which the ammonium and choline salts are excluded.)

These earlier TRI 50c salts were made via the general method described in Comparative Example A of this specification. Impurity I has the following structure:

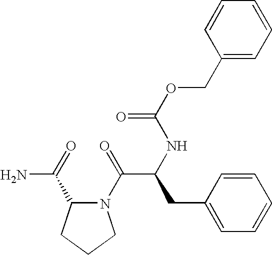

For example, an HPLC chromatogram, prepared using a reverse phase method more particularly described in Example 5, produced the following data for the monosodium salt of TRI 50c, the TRI 50c having been made by a procedure following the general method of Comparative Example A:

| | | RT | | | | | |
|---|---|---|---|---|---|---|---|
| | Name | (min) | Area | Height | Amount | Units | % Area |
| 1 | Benzal- | 6.145 | 2487 | 224 | | | 0.39 |
| | dehyde | | | | | | |
| 2 | Impurity | 11.022 | 6379 | 539 | | | 1.00 |
| | I | | | | | | |
| 3 | TRI50c | 11.679 | 628872 | 51108 | 946,063 | ug/mL | 98.61 |

Attempts to purify salts contaminated with Impurity I were not successful, and it appeared that, for example, Impurity I was generated from the salts in HPLC columns.

Relative chiral purity of salts made via the general procedure of Comparative Example A was achieved by resolving by HPLC the pinacol ester of TRI 50c, designated TRI 50b, and converting the thus-resolved TRI 50b into the salts. Such an HPLC procedure is not acceptable for normal commercial drug production.

It has further been found that the prior art synthesis summarised earlier under the heading “Aminoboronate Procedure” results, when applied to the synthesis of TRI 50c or an ester thereof, in formation of an impurity designated Impurity IV:

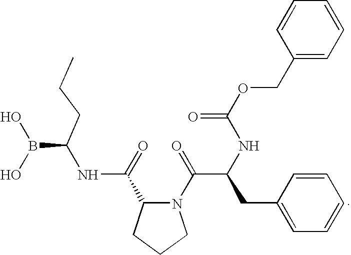

Attempts to separate Impurity IV from TRI 50c have not succeeded. The same applies to TRI 50c salts and esters and the corresponding salts and esters of Impurity IV. No purification technique which has been tried can prevent the presence of Impurity IV if said prior art synthesis is used.

The Methods

Amongst other things, the present disclosure addresses the problems of controlling C—B bond cleavage in organoboronic compounds as well as providing chirally purified salts of TRI 50c and other organoboronic acids on a commercial scale.

It has also been found that chirally-selective precipitation can be used to recover organoboronic acids in high purity.

Thus C—B bond cleavage (and hence in particular generation of Impurity I) may be controlled by:
Selection of acetonitrile as a solvent, where a solvent is required in processing and acetonitrile has the necessary solvation power; in particular acetonitrile is selected in process where a polar solvent is desirable or necessary.
Avoiding excessive contact with water.

In terms of TRI 50c salt production, therefore, the disclosure includes processes comprising one, two or three of the following features:
(i) resolution of the (R,S,S) and (R,S,R) epimers of TRI 50c by chirally selective precipitation using diethanolamine and conveniently, but not necessarily, using as starting material TRI 50c in the form of an ester, for example the pinacol ester;
(ii) control of the duration and/or conditions of hydrolysis of TRI 50c diethanolamine ester, for example as obtained by such precipitation, to control C—B bond breakage;
(iii) use of acetonitrile as solvent for TRI 50c, for example as obtained by such hydrolysis, for the purposes of reacting the TRI 50c with a base to form the salt. Another favourable solvent can be tetrahydrofuran.

As an optional, or even stand-alone, fourth feature, TRI 50c salts may be dried by azeodrying using acetonitrile.

The above four features, or any one, two or three of them, may be applied to the manufacture and processing of other boronic compounds, particularly acids of formula (I) and their derivatives (e.g. esters and salts), as well as other boropeptides.

The disclosure provides in one aspect, therefore, the use of diethanolamine to resolve by selective precipitation the diastereomers of boronic acids of formula (Ia): 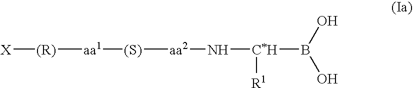
where:
X is H (to form NH₂) or an amino-protecting group;
aa¹ is an amino acid of (R) configuration selected from Phe, Dpa and wholly or partially hydrogenated analogues thereof;
aa² is an imino acid of (S) configuration having from 4 to 6 ring members;
R¹ is a group of the formula —(CH₂)ₛ-Z, where s is 2, 3 or 4 and Z is —OH, —OMe, —OEt or halogen selected from F, Cl, Br or I,
and where C* is a chiral centre.

The starting material may be an acid (Ia) or a derivative thereof capable of forming a diethanolamine ester of the boronic acid. The precipitation selects acids having a chiral centre C* of (R) configuration as precipitate. The precipitate may be recovered and converted to the corresponding boronic acid or a salt thereof. The salt may be made into a pharmaceutical formulation. In practice, the starting material may contain trace amounts of acid in which the fragment aa¹-aa² is not of (R,S) configuration, e.g. it may be at least 99.5% (R,S), and in some cases at least 99.7% (R,S).

For optimised chiral purity and yield, the diethanolamine may be used in an amount of about 1.25±0.1 equivalents based on initial equivalents of boronic acid having a chiral centre C* of (R) configuration.

The initial boronic acid or acid derivative may for example comprise from 50% to 60% molecules having chiral centre C* of (R)-configuration and from 40% to 50% molecules having chiral centre C* of (S)-configuration.

The method opens the way to commercialisation of the boronic acids (Ia) and their derivatives, particularly salts, as pharmaceuticals. Commercial scale products and activities using the boronic acids (Ia) and their derivatives are therefore provided.

In one embodiment, there is provided a process for separating diastereomers of a boronic acid of formula (Ia), comprising:
combining in diethylether solution (A) a boronic species selected from the boronic acid (I) and its esters, the boronic species including molecules having a chiral centre C* of (R) configuration and molecules having a chiral centre C* of (S) configuration, and (B) diethanolamine, the diethanolamine being in an amount of about 1.25±0.1 equivalents based on the boronic species in which the chiral centre C* is of (R) configuration, and mixing to form a mixture;
causing or allowing the boronic species and the diethanolamine to react until a precipitate forms; and
recovering the precipitate.

When the starting material is an ester, it may be an ester of the boronic acid with an alcohol selected from the group consisting of alcohols whose sole potential electron donor heteroatoms are oxygens which, in the boronic ester, correspond to the oxygens of the ester functional group.

In some methods, the diethanolamine is in an amount of from 1.2 to 1.3 equivalents based on the boronic species in which chiral centre C* is of (R) configuration.

There are included processes in which the boronate species is an ester of the boronic acid and a diol, in particular a diol which is not sterically hindered. As exemplary diols may be mentioned pinacol, neopentylglycol, 1,2-ethanediol, 1,2-propanediol, 1,3-propanediol, 2,3-butanediol, 1,2-diisopropylethanediol, or 5,6-decanediol. A particular diol is pinacol.

The boronic species and the diethanolamine may be caused to react by heating the mixture to an elevated temperature, for example the mixture may be refluxed. e.g. for at least 10 hours.

The precipitate may be recovered by filtration. The recovered precipitate may be washed with diethylether. The recovered precipitate, after washing if such takes places, may be dissolved in a solvent selected from CH₂Cl₂ and CHCl₃ and reprecipitated by combining the resulting solution with diethylether. A particular solvent is CH₂Cl₂.

The recovered precipitate (consisting substantially exclusively of an adduct between diethanolamine and the (R,S,R) isomer of the acid) may be converted to the acid of formula (Ia), suitably by hydrolysis, for example by dissolving the precipitate in an organic solvent selected from e.g. halohydrocarbons and combinations thereof, agitating the resulting solution with an aqueous liquid, e.g. an aqueous acid having a pH of below 3, whereby the dissolved precipitate is converted to the formula (Ia) acid, and recovering the formula (Ia) acid by evaporation. The organic solvent may be CH₂Cl₂ or CHCl₃. A particular solvent is CH₂Cl₂. In some processes, organic solvent is further evaporated from the recovered formula (Ia) acid.

The disclosure includes methods in which an ester (particularly a diethanolamine ester) of a organoboronic acid, for example an aminoboronate or peptide boronate such as, e.g. a boronic acid of formula (I) or formula (Ia), is hydrolysed in a manner which controls C—B bond cleavage. In particular, this involves limiting the period of hydrolysis at the selected temperature. In the case of diethanolamine ester hydrolysis, the hydrolysis is suitably carried out at room temperature, or less, for a period not exceeding about 30 minutes, e.g. not exceeding about 20 minutes, and optimally of about 20 minutes. In more general terms, the duration of hydrolysis of the ester is limited to avoid substantial C—B bond breakage, i.e. substantially to avoid generation of the degradation product resulting from such bond breakage. By way of example, the product acid (or a salt produced therefrom) may contain at most about 0.5% of such degradation product by weight of the total product, e.g. less than about 0.3 wt % and often less than about 0.2 wt %. The content of C—B bond degradation product may be about 0.1 wt % or less. In particular instances, there is no more than about 0.05% degradation product as determined by reverse phase HPLC (see Example 43 below). Included are boronic acids and their base addition salts in which there is no C—B degradation product detectable by the HPLC technique of Example 43, or about such amount; of course, hydrolysis methods which result in boronic acids having such a level of purity are also included. In the case of TRI 50c and its salts, the degradation product of C—B bond cleavage of which it is substantially free is Impurity I; base addition salts of TRI 50c have been prepared in which Impurity I was not detected with the initial HPLC analysis.

The disclosure includes methods in which an ester of a boronic acid (I) or formula (Ia), particularly a diethanolamine ester, is hydrolysed in a manner which controls C—B bond cleavage. In particular, this involves limiting the period of hydrolysis at the selected temperature. In the case of diethanolamine ester hydrolysis, the hydrolysis is suitably carried out at room temperature, or less, for a period not exceeding about 30 minutes, e.g. not exceeding about 20 minutes, and optimally of about 20 minutes.

Thus the recovered precipitate referred to in the last paragraph but one may be hydrolysed using an aqueous acid, particularly 2% hydrochloric acid or another mineral acid of similar pH, for no more than about 30 minutes at about room temperature, or less. Suitably, the precipitate is dissolved in a non-nucleophilic organic solvent (e.g. a halohydrocarbon or halohydrocarbon mixture for example CH₂Cl₂) and the resulting solution is contacted with the aqueous acid for a period as previously described. The precipitate is thereby hydrolysed to form the free acid of formula (I) or (Ia), which remains in the organic solvent. The organic solvent may be separated from the aqueous medium and then evaporated to obtain solid acid of formula (I) or (Ia).

There are included processes in which a formula (I) or formula (Ia) acid, for example obtained as described in the preceding paragraph, is dried. In a class of processes, the formula (I) acid is dried when it is in the organic solvent by contacting the solvent with a hygroscopic solid.

Included are processes in which the formula (I) or formula (Ia) acid, when in the organic solvent, is washed with an aqueous ammonium salt.

Chirally purified boronic acid may be converted to a pharmaceutically acceptable base addition salt thereof, in particular by dissolving the acid in acetonitrile, combining the resultant solution with an aqueous solution or suspension of a pharmaceutically acceptable base, and causing or allowing the base and the acid to react, then evaporating to dryness to obtain an evaporation residue. The step of causing or allowing the acid and the base to react may comprise agitating the combination of the acetonitrile solution of the acid and the aqueous solution or suspension of the base at a temperature of not more than 35° C. and often of not more than 30° C., e.g. not more than 25° C.; an optimal temperature is room temperature, in which case a reaction time of about 2 hours might be appropriate. The process may further comprise:
(i) redissolving the evaporation residue in acetonitrile and evaporating the resulting solution to dryness; and
(ii) repeating step (i) as often as necessary to obtain a dry evaporation residue.

In some processes the dry evaporation residue is dissolved in acetonitrile or tetrahydrofuran to form a solution, and the solution is combined with (e.g. slowly added to, at a rate sufficiently slow to avoid lump formation) a 3:1 to 1:3 v/v mixture of diethylether and an aliphatic or cycloaliphatic solvent to form a precipitate, said solution being added to the diethylether/(cyclo)aliphatic solvent mixture in a ratio (solution:mixture) of from 1:5 to 1:15 v/v. The precipitate is recovered and some or substantially all remaining solvent is removed from the recovered precipitate whilst maintaining the temperature at no more than 35° C., e.g. is removed under reduced pressure. Included are processes in which the temperature at the start of the drying process is about 10° C. and is increased during the process to 35° C. The aliphatic or cycloaliphatic solvent may have 6, 7 or 8 carbon atoms; the solvent may be an alkane, for example an n-alkane, e.g. n-heptane. Some reactions may be carried out at ambient temperature, which may e.g. be 15-30° C., e.g. 20-30° C.; sometimes ambient temperature may be room temperature.

The salts produced by the invention may contain a trace amount of the aliphatic or cycloaliphatic solvent, e.g. an amount of less than 0.1%, particularly less than 0.01%, for example an amount of about 0.005%.

In the process for making the salt, the base may comprise a cation of valency n and be used in a stoichiometry (boronic acid:base) of about n:1. In particular processes, the base is an alkali metal or alkaline earth metal base, for example an alkali metal hydroxide or an alkaline earth metal hydroxide. As one base may be mentioned sodium hydroxide. As another base may be mentioned calcium hydroxide. The disclosure includes processes in which the base is sodium hydroxide and the dry evaporation residue is dissolved in acetonitrile. The disclosure includes processes in which the base is calcium hydroxide and the dry evaporation residue is dissolved in tetrahydrofuran.

The disclosure is not limited as to the method by which the boronic acids of Formula (I) or Formula (Ia) are obtained (for example as an ester thereof). However, in one class of subject matter, the Formula (I) acid has an R¹ group of the formula —(CH₂)ₛ—O—R³ in which R³ is methyl or ethyl and s is independently 2, 3 or 4, and the Formula (I) acid is prepared via an intermediate of Formula (XXV):
(HO)₂B—(CH₂)ₛ—O—R³ (XXV),
which intermediate is made by reaction between a borate ester and a suitable 1-metalloalkoxyalkane.

A novel aspect of the disclosure comprises the Formula (XXV) intermediates.

The Formula (XXV) intermediates may be made by reacting a 1-metalloalkoxyalkane, where the alkoxyalkane is of the formula —(CH₂)ₛ—O—R³, with a borate ester to form a compound of Formula (XXV).

It will be appreciated that the above method provides a general procedure for making alkoxyalkylboronic acids, which may be presented by the formula R^{Z}—O—R^{Y}—B(OH)₂. Such alkoxyalkylboronic acids may be converted to aminoboronates, and the aminoboronates may be derivatised at their amino group to form an amide bond linked to another moiety. In other words, the aminoboronates may be converted to boropeptides. The method will now be described further with non-limiting reference to compounds of Formula (XXV).

The starting materials for the reaction may be a metalloalkoxyalkane, e.g. a Grignard reagent, obtainable from 1-haloalkoxyalkane of the formula Hal-(CH₂)ₛ—O—R³ (where Hal is a halogen) and a borate ester. The metal is in particular magnesium. Another metal is lithium, in which case the metallo reagent may be prepared by reacting the 1-haloalkoxyalkane with butyl lithium. Where the method includes preparation of the metallo reagent from the haloalkoxyalkane, the haloalkoxyalkane may be a chloroalkoxyalkane; the corresponding bromo compounds may also be used. To make a Grignard reagent, magnesium may be reacted with the haloalkoxyalkane.

Suitable borate esters are esters of mono- and di-functional alcohols (e.g. of EtOH, MeOH, BuOH, pinacol, glycol, pinanediol etc). For example, the ester may be of the formula B(OR^{a})(OR^{b})(OR^{c}) where R^{a}, R^{b} and R^{c} and C₁-C₄ alkyl and may be the same as each other.

An exemplary procedure for making a Formula (XXV) intermediate, illustrated with reference to methoxypropane as the alkoxyalkane species, is:

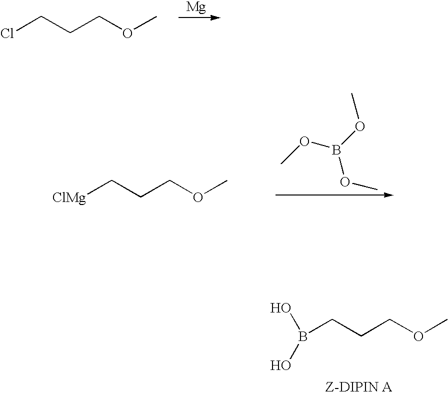

The reactions are suitably carried out in an organic solvent, e.g. THF.

The above-described procedure for making alkoxyalkylboronic acids avoids generation of Impurity IV (see above), or its analogues in those cases where the end product is not TRI 50c or a derivative (salt, ester etc) thereof. The procedure therefore provides a unique route to making TRI 50c, its esters and salts, uncontaminated by Impurity IV, and for making other aminoboronic acids which are substituted α- to the boron by an alkoxyalkyl group and are uncontaminated by impurities analogous to Impurity IV.

An alkoxyalkylboronic acid, i.e. a compound which may be represented by the formula R^{Z}—O—R^{Y}—B(OH)₂, may be converted to an aminoboronic compound, for example a boropeptide, by any suitable procedure, e.g. one known in the art. A reaction scheme for making alkoxyalkylboronic acids into aminoboronates, and for converting aminoboronates into peptide boronates is illustrated with reference to synthesis of TRI 50c at the start of the Examples of this specification. The reaction scheme may be modified as desired, e.g.: diethanolamine precipitation and subsequent steps may be omitted, and/or reagent substitutions may be made. For example, pinacol may be replaced by another diol. LDA is a non-nucleophilic strong base and may be replaced by another such base. Other examples include, but are not limited to, lithium diisopropylamide, lithium 2,2,6,6-tetramethylpiperidine, 1-lithium 4methylpiperazide, 1,4-dilithium piperazide, lithium bis(trimethylsilyl) amide, sodium bis(trimethylsilyl)amide, potassium bis(trimethylsilyl)amide, isopropyl magnesium chloride, phenyl magnesium chloride, lithium diethylamide, and potassium tert-butoxide. The reactions may be carried out in any suitable solvent: where n-heptane is used in the Examples, it may be replaced by another inert non-polar solvent, e.g. another aliphatic or cycloaliphatic solvent, for example an alkane, e.g. an n-alkane.

Thus, the disclosure includes a process for making an aminoboronate of Formula (XXI) 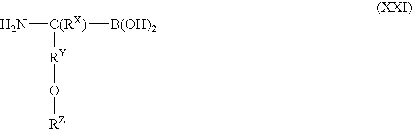
wherein
R^{X} is H or a substituent which does not prevent synthesis;
R^{Y} is alkylene; and
R^{Z} is alkyl,
the process comprising reacting a 1-metalloalkoxyalkane with a borate ester to form a boronic acid of the formula R^{Z}—O—R^{Y}—B(OH)₂, esterifying the acid, contacting the esterified acid with CH₂Cl₂ and ZnCl₂ in the presence of a strong base, contacting the resultant product with LiHMDS and in turn contacting the resultant product with hydrogen chloride.

The product is free of contaminant of Formula (XXII):
H₂N—C(R^{X})(R^{Y})—B(OH)₂ (XXII).

The aminoboronate (XXI) may be reacted with an amino acid or peptide (which in either case may be suitably protected) to form a peptide boronate. In general terms, therefore, the disclosure includes peptidoboronic acids of Formula (XXIII): 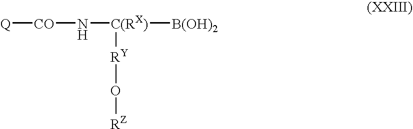
Q-CO comprises at least an amino acid residue;
R^{X} is H or a substituent which does not prevent synthesis;
R^{Y} is alkylene;
R^{Z} is alkyl,
   which organoboronic acid is free of an impurity of Formula (XXIV):

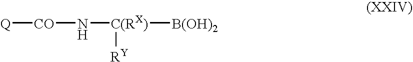

The disclosure further includes derivatives of Formula (XXIII) acids (e.g. acid or base addition salts, esters) which are free of Formula (XXIV) impurity and derivatives thereof.

The exact identity of R^{Y} and R^{Z} is dependent on the identity of the end product, and not part of the process or its benefits.

It will be appreciated from the aforegoing that the above described methods may be used in the manufacture of organoboronic acids salts as described. It is not necessary for sequential steps to be carried out as one operation or at the same site: they may be performed in this way or different processes (different parts of the overall synthesis) may be distributed in time and/or space. Particular end product salts are monosodium, monolithium, hemicalcium and hemimagnesium salts, for example of TRI 50c.

Generally, the reactions may suitably be carried out with a non-nucleophilic solvent. Where a nucleophilic solvent is present, minimum contact is preferred, for example in the case of hydrolysis of diethanolamine esters.

The Products

The products of the disclosure include inter alia boronic acids, diethanolamine esters and salts obtainable by (having the characteristics of a product obtained by) the disclosed methods. Also included are products obtained directly or indirectly by the disclosed methods.

Particular products of the disclosure are base addition salts of a boronic acid of formula (I) having the chiral purity of such salt when prepared by a method described herein.

Included are esters of boronic acids of formula I (for example, diethanolamine esters), the free acids of formula (I) and salts of the free acid which comprise the (R,S,R) diastereomer in a diastereomeric excess over the (R,S,S) diastereomer of about 95% or more. The (R,S,R) isomer may be in a diastereomeric excess of at least about 98%, and optionally of about 99% or more, e.g. about 99.5% or more. Further included are salts having a diastereomeric excess [(R,S,R) over (R,S,S)] of about 99.5% or more and purity as measured by % HPLC peak area of at least 95% when determined by the method of Example 5; in particular, the salt is a metal salt of TRI 50c, e.g. an alkali metal or alkaline earth metal salt.

Other products are base addition salts of a boronic acid of formula (I) having the purity of such salt when prepared by a method described herein.

Product identities will be apparent from the preceding description and the following examples. In addition, products of the disclosure are described in the claims. Of particular note are the data in Example 5, indicating that the processes of the disclosure can remarkably achieve end product salts free of impurities detectable by the described HPLC method. In other instances, the salts are substantially free of impurities, e.g. at least 98% pure, more usually at least 99% pure, e.g. at least 99.5% pure, in terms of reverse phase (RP) HPLC percentage peak area. Salts may at least 99.3%, 99.4%, 99.5% 99.6%, 99.7%, 99.8% or 99.9% pure, in terms of reverse phase (RP) HPLC percentage peak area. Suitable RP HPLC procedures comply with reference 1 and/or reference 2 and/or reference 3 of Example 5. Included also are products at least substantially free of Impurity I and analogues, products free of Impurity IV and analogues, and products containing small traces of non-polar solvent, e.g. n-heptane. The trace amount of non-polar solvent may be less than 0.2%, 0.1%, 0.05%, 0.01% or 0.005% as determined by GC-headspace chromatography.

Also to be mentioned is Cbz-(R)-Phe-(S)-Pro-(R)-Mpg-B(OH)₂, and the salts thereof, substantially free of Impurity I, i.e. the compound:

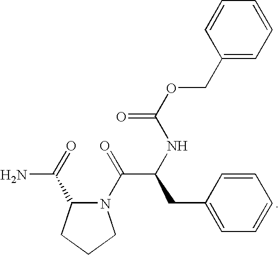

A further class of compounds comprises Cbz-(R)-Phe-(S)-Pro-(R)-Mpg-B(OH)₂, and the esters and salts thereof, substantially free of Impurity IV, i.e. the compound:

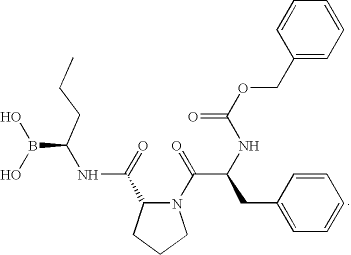

Included also are salts containing less than 410 ppm acetonitrile.

Some salts contain impurities of less than 10,000 ppm, 5000 ppm, 1000 ppm, or 500 ppm.

Use of the Products of the Disclosure

The formula (I) acids and their salts are thrombin inhibitors. They are therefore useful for inhibiting thrombin. The disclosure therefore provides compounds which have potential for controlling haemostasis and especially for inhibiting coagulation, for example in treatment or prevention of myocardial infarction. The medical use of the compounds may be prophylactic (including to treat thrombosis as well as to prevent occurrence of thrombosis) as well as therapeutic (including to prevent re-occurrence of thrombosis or secondary thrombotic events).

The salts may be employed when an anti-thrombogenic agent is needed. They are thus indicated in the treatment or prophylaxis of thrombosis and hypercoagulability in blood and tissues of animals including man. The term “thrombosis” includes inter alia atrophic thrombosis, arterial thrombosis, cardiac thrombosis, coronary thrombosis, creeping thrombosis, infective thrombosis, mesenteric thrombosis, placental thrombosis, propagating thrombosis, traumatic thrombosis and venous thrombosis.

It is known that hypercoagulability may lead to thromboembolic diseases.

Examples of venous thromboembolism which may be treated or prevented with thrombin-inhibitory compounds of the disclosure include obstruction of a vein, obstruction of a lung artery (pulmonary embolism), deep vein thrombosis, thrombosis associated with cancer and cancer chemotherapy, thrombosis inherited with thrombophilic diseases such as Protein C deficiency, Protein S deficiency, antithrombin III deficiency, and Factor V Leiden, and thrombosis resulting from acquired thrombophilic disorders such as systemic lupus erythematosus (inflammatory connective tissue disease). Also with regard to venous thromboembolism, thrombin-inhibitory compounds of the disclosure are useful for maintaining patency of indwelling catheters.

Examples of cardiogenic thromboembolism which may be treated or prevented with thrombin-inhibitory compounds of the disclosure include thromboembolic stroke (detached thrombus causing neurological affliction related to impaired cerebral blood supply), cardiogenic thromboembolism associated with atrial fibrillation (rapid, irregular twitching of upper heart chamber muscular fibrils), cardiogenic thromboembolism associated with prosthetic heart valves such as mechanical heart valves, and cardiogenic thromboembolism associated with heart disease.

Examples of conditions involving arterial thrombosis include unstable angina (severe constrictive pain in chest of coronary origin), myocardial infarction (heart muscle cell death resulting from insufficient blood supply), ischemic heart disease (local ischemia due to obstruction (such as by arterial narrowing) of blood supply), reocclusion during or after percutaneous transluminal coronary angioplasty, restenosis after percutaneous transluminal coronary angioplasty, occlusion of coronary artery bypass grafts, and occlusive cerebrovascular disease. Also with regard to arterio-venous (mixed) thrombosis, anti-thrombotic compounds of the disclosure are useful for maintaining patency in arteriovenous shunts.

Other conditions associated with hypercoagulability and thromboembolic diseases which may be mentioned inherited or acquired deficiencies in heparin cofactor II, circulating antiphospholipid antibodies (Lupus anticoagulant), homocysteinemia, heparin induced thrombocytopenia and defects in fibrinolysis.

Particular uses which may be mentioned include the therapeutic and/or prophylactic treatment of venous thrombosis and pulmonary embolism. Preferred indications envisaged for the thrombin-inhibitory products of the disclosure (notably the salts of TRI 50c) include:
Prevention of venous thromboembolic events (e.g. deep vein thrombosis and/or pulmonary embolism). Examples include patients undergoing orthopaedic surgery such as total hip replacement, total knee replacement, major hip or knee surgery; patients undergoing general surgery at high risk for thrombosis, such as abdominal or pelvic surgery for cancer; and in patients bedridden for more than 3 days and with acute cardiac failure, acute respiratory failure, infection.
Prevention of thrombosis in the haemodialysis circuit in patients, in patients with end stage renal disease.
Prevention of cardiovascular events (death, myocardial infarction, etc) in patients with end stage renal disease, whether or not requiring haemodialysis sessions.
Prevention of venous thrombo-embolic events in patients receiving chemotherapy through an indwelling catheter.
Prevention of thromboembolic events in patients undergoing lower limb arterial reconstructive procedures (bypass, endarteriectomy, transluminal angioplasty, etc).
Treatment of venous thromboembolic events.
Prevention of cardiovascular events in acute coronary syndromes (e.g. unstable angina, non Q wave myocardial ischaemia/infarction), in combination with another cardiovascular agent, for example aspirin (acetylsalicylic acid; aspirin is a registered trade mark in Germany), thrombolytics (see below for examples), antiplatelet agents (see below for examples).
Treatment of patients with acute myocardial infarction in combination with acetylsalicylic acid, thrombolytics (see below for examples).

The thrombin inhibitors of the disclosure are thus indicated both in the therapeutic and/or prophylactic treatment of all the aforesaid disorders.

In one method, the products of the disclosure are used for the treatment of patients by dialysis, by providing the product in the dialysis solution, as described in relation to other thrombin inhibitors in WO 00/41715, which is incorporated herein by reference. The disclosure therefore includes dialysing solutions and dialysing concentrates which comprise a described thrombin-inhibitory product, as well as a method of treatment by dialysis of a patient in need of such treatment, which method comprises the use of a dialysing solution including a low molecular weight thrombin inhibitor. Also included is the use of a disclosed anti-thrombotic product for the manufacture of a medicament for the treatment by dialysis of a patient, in which the anti-thrombotic product of the disclosure is provided in the dialysing solution.

In another method, the thrombin-inhibitory products of the disclosure are used to combat undesirable cell proliferation, as described in relation to other thrombin inhibitors in WO 01/41796, which is incorporated herein by reference. The undesirable cell proliferation is typically undesirable hyperplastic cell proliferation, for example proliferation of smooth muscle cells, especially vascular smooth muscle cells. The described thrombin-inhibitory products particularly find application in the treatment of intimal hyperplasia, one component of which is proliferation of smooth muscle cells. Restenosis can be considered to be due to neointimal hyperplasia; accordingly intimal hyperplasia in the context of the disclosure includes restenosis.

The products of the described thrombin-inhibitory are also contemplated for the treatment of ischemic disorders. More particularly, they may be used in the treatment (whether therapeutic or prophylactic) of an ischemic disorder in a patient having, or at risk of, non-valvular atrial fibrillation (NVAF) as described in relation to other thrombin inhibitors in WO 02/36157, which is incorporated herein by reference. Ischemic disorders are conditions whose results include a restriction in blood flow to a part of the body. The term will be understood to include thrombosis and hypercoagulability in blood, tissues and/or organs. Particular uses that may be mentioned include the prevention and/or treatment of ischemic heart disease, myocardial infarction, systemic embolic events in e.g. the kidneys or spleen, and more particularly of cerebral ischemia, including cerebral thrombosis, cerebral embolism and/or cerebral ischemia associated with non-cerebral thrombosis or embolism (in other words the treatment (whether therapeutic or prophylactic) of thrombotic or ischemic stroke and of transient ischemic attack), particularly in patients with, or at risk of, NVAF.

The thrombin-inhibitory products of the disclosure are also contemplated for the treatment of rheumatic/arthritic disorders, as described in relation to other thrombin inhibitors in WO 03/007984, which is incorporated herein by reference. Thus, the products may be used in the treatment of chronic arthritis, rheumatoid arthritis, osteoarthritis or ankylosing spondylitis

Moreover, the products are expected to have utility in prophylaxis of re-occlusion (i.e. thrombosis) after thrombolysis, percutaneous trans-luminal angioplasty (PTA) and coronary bypass operations; the prevention of re-thrombosis after microsurgery and vascular surgery in general. Further indications include the therapeutic and/or prophylactic treatment of disseminated intravascular coagulation caused by bacteria, multiple trauma, intoxication or any other mechanism; anticoagulant treatment when blood is in contact with foreign surfaces in the body such as vascular grafts, vascular stents, vascular catheters, mechanical and biological prosthetic valves or any other medical device; and anticoagulant treatment when blood is in contact with medical devices outside the body such as during cardiovascular surgery using a heart-lung machine or in haemodialysis.

The products of the disclosed thrombin-inhibitory products are further indicated in the treatment of conditions where there is an undesirable excess of thrombin without signs of hypercoagulability, for example in neurodegenerative diseases such as Alzheimer's disease. In addition to its effects on the coagulation process, thrombin is known to activate a large number of cells (such as neutrophils, fibroblasts, endothelial cells and smooth muscle cells). Therefore, the compounds of the disclosure may also be useful for the therapeutic and/or prophylactic treatment of idiopathic and adult respiratory distress syndrome, pulmonary fibrosis following treatment with radiation or chemotherapy, septic shock, septicaemia, inflammatory responses, which include, but are not limited to, edema, acute or chronic atherosclerosis such as coronary arterial disease, cerebral arterial disease, peripheral arterial disease, reperfusion damage, and restenosis after percutaneous trans-luminal angioplasty (PTA).

The salts may also be useful in the treatment of pancreatitis.

The salts described herein are further considered to be useful for inhibiting platelet procoagulant activity. The disclosure provides a method for inhibiting platelet pro-coagulant activity by administering a salt of a boronic acid described herein to a mammal at risk of, or suffering from, arterial thrombosis, particularly a human patient. Also provided is the use of such salts for the manufacture of medicaments for inhibiting platelet procoagulant activity.

The use at least of products comprising an acid of formula (II) or salt thereof as inhibitors of platelet pro-coagulant activity is predicated on the observation that such boronic acids are indicated to be effective at inhibiting arterial thrombosis as well as venous thrombosis.

Indications involving arterial thrombosis include acute coronary syndromes (especially myocardial infarction and unstable angina), cerebrovascular thrombosis and peripheral arterial occlusion and arterial thrombosis occurring as a result of atrial fibrillation, valvular heart disease, arterio-venous shunts, indwelling catheters or coronary stents. Accordingly, in another aspect the disclosure provides a method of treating a disease or condition selected from this group of indications, comprising administering to a mammal, especially a human patient, a salt of the disclosure. The disclosure includes products for use in an arterial environment, e.g. a coronary stent or other arterial implant, having a coating which comprises a salt of the disclosure.

The salts of the disclosure may be used prophylactically to treat an individual believed to be at risk of suffering from arterial thrombosis or a condition or disease involving arterial thrombosis or therapeutically (including to prevent re-occurrence of thrombosis or secondary thrombotic events).

The disclosure therefore includes the use of selective thrombin inhibitors (organoboronic acid salts) described herein for treatment of the above disorders by prophylaxis or therapy as well as their use in pharmaceutical formulations and the manufacture of pharmaceutical formulations.

Administration and Pharmaceutical Formulations

The salts may be administered to a host, for example, in the case where the drug has anti-thrombogenic activity, to obtain an anti-thrombogenic effect. In the case of larger animals, such as humans, the compounds may be administered alone or in combination with pharmaceutically acceptable diluents, excipients or carriers. The term “pharmaceutically acceptable” includes acceptability for both human and veterinary purposes, of which acceptability for human pharmaceutical use is preferred.

The salts of the disclosure may be combined and/or co-administered with any cardiovascular treatment agent. There are large numbers of cardiovascular treatment agents available in commercial use, in clinical evaluation and in pre-clinical development, which could be selected for use with a product of the disclosure for the prevention of cardiovascular disorders by combination drug therapy. Such agent can be one or more agents selected from, but not limited to several major categories, namely, a lipid-lowering drug, including an IBAT (ileal Na⁺/bile acid cotransporter) inhibitor, a fibrate, niacin, a statin, a CETP (cholesteryl ester transfer protein) inhibitor, and a bile acid sequestrant, an anti-oxidant, including vitamin E and probucol, a IIb/IIIa antagonist (e.g. xemilofiban and orbofiban), an aldosterone inhibitor (e.g. spirolactone and epoxymexrenone), an adenosine A2 receptor antagonist (e.g. losartan), an adenosine A3 receptor agonist, a beta-blocker, acetylsalicylic acid, a loop diuretic and an ACE (angiotensin converting enzyme) inhibitor.

The salts of the disclosure may be combined and/or co-administered with any antithrombotic agent with a different mechanism of action, such as the antiplatelet agents acetylsalicylic acid, tidopidine, clopidogrel, thromboxane receptor and/or synthetase inhibitors, prostacyclin mimetics and phosphodiesterase inhibitors and ADP-receptor (P₂ T) antagonists.

The products of the disclosure may further be combined and/or co-administered with thrombolytics such as tissue plasminogen activator (natural, recombinant or modified), streptokinase, urokinase, prourokinase, anisoylated plasminogen-streptokinase activator complex (APSAC), animal salivary gland plasminogen activators, and the like, in the treatment of thrombotic diseases, in particular myocardial infarction.

The salts of the disclosure may be combined and/or co-administered with a cardioprotectant, for example an adenosine A1 or A3 receptor agonist.

There is also provided a method for treating an inflammatory disease in a patient that comprises treating the patient with a product of the disclosure and an NSAID, e.g., a COX-2 inhibitor. Such diseases include but are not limited to nephritis, systemic lupus, erythematosus, rheumatoid arthritis, glomerulonephritis, vasculitis and sarcoidosis. Accordingly, the anti-thrombotic salts of the disclosure may be combined and/or co-administered with an NSAID.

Typically, therefore, the salts described herein may be administered to a host to obtain a thrombin-inhibitory effect, or in any other thrombin-inhibitory or anti-thrombotic context mentioned herein.

Actual dosage levels of active ingredients in the pharmaceutical compositions of this disclosure may be varied so as to obtain an amount of the active compound(s) that is effective to achieve the desired therapeutic response for a particular patient, compositions, and mode of administration. The selected dosage level will depend upon the activity of the particular compound, the severity of the condition being treated and the condition and prior medical history of the patient being treated. However, it is within the skill of the art to start doses of the compound at levels lower than required for to achieve the desired therapeutic effect and to gradually increase the dosage until the desired effect is achieved.

For example, it is currently contemplated that, in the case of oral administration of salts of TRI 50c, the salts might for instance be administered in an amount of from 0.5 to 2.5 mg/Kg twice daily, calculated as TRI 50c. Other salts might be administered in equivalent molar amounts. The disclosure is not limited to administration in such quantities or regimens and includes dosages and regimens outside those described in the previous sentence.

According to a further aspect of the disclosure there is provided an oral pharmaceutical formulation including a product of the disclosure, in admixture with a pharmaceutically acceptable adjuvant, diluent or carrier.

Solid dosage forms for oral administration include capsules, tablets (also called pills), powders and granules. In such solid dosage forms, the active compound is typically mixed with at least one inert, pharmaceutically acceptable excipient or carrier such as sodium citrate or dicalcium phosphate and/or one or more: a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules and tablets, the dosage form may also comprise buffering agents. Solid compositions of a similar type may also be employed as fillers in soft and hard-filled gelatin capsules using such excipients as lactose or milk sugar as well as high molecular weight polyethylene glycol, for example.

Suitably, the oral formulations may contain a dissolution aid. The dissolution aid is not limited as to its identity so long as it is pharmaceutically acceptable. Examples include nonionic surface active agents, such as sucrose fatty acid esters, glycerol fatty acid esters, sorbitan fatty acid esters (e.g., sorbitan trioleate), polyethylene glycol, polyoxyethylene hydrogenated castor oil, polyoxyethylene sorbitan fatty acid esters, polyoxyethylene alkyl ethers, methoxypolyoxyethylene alkyl ethers, polyoxyethylene alkylphenyl ethers, polyethylene glycol fatty acid esters, polyoxyethylene alkylamines, polyoxyethylene alkyl thioethers, polyoxyethylene polyoxypropylene copolymers, polyoxyethylene glycerol fatty acid esters, pentaerythritol fatty acid esters, propylene glycol monofatty acid esters, polyoxyethylene propylene glycol monofatty acid esters, polyoxyethylene sorbitol fatty acid esters, fatty acid alkylolamides, and alkylamine oxides; bile acid and salts thereof (e.g., chenodeoxycholic acid, cholic acid, deoxycholic acid, dehydrocholic acid and salts thereof, and glycine or taurine conjugate thereof); ionic surface active agents, such as sodium laurylsulfate, fatty acid soaps, alkylsulfonates, alkylphosphates, ether phosphates, fatty acid salts of basic amino acids; triethanolamine soap, and alkyl quaternary ammonium salts; and amphoteric surface active agents, such as betaines and aminocarboxylic acid salts.

In the case of oral administration, the compounds may be administered in a form which prevents the salt of the disclosure from contact with the acidic gastric juice, such as enterically coated formulations, which thus prevent release of the salt of the disclosure until it reaches the duodenum.

The enteric coating is suitably made of carbohydrate polymers or polyvinyl polymers, for example. Examples of enteric coating materials include, but are not limited to, cellulose acetate phthalate, cellulose acetate succinate, cellulose hydrogen phthalate, cellulose acetate trimellitate, ethyl cellulose, hydroxypropyl-methylcellulose phthalate, hydroxypropylmethylcellulose acetate succinate, carboxymethyl ethylcellulose, starch acetate phthalate, amylose acetate phthalate, polyvinyl acetate phthalate, polyvinyl butyrate phthalate, styrene-maleic acid copolymer, methyl-acrylate-methacrylic acid copolymer (MPM-05), methylacrylate-methacrylic acid-methylmethacrylate copolymer (MPM-06), and methylmethacrylate-methacrylic acid co-polymer (Eudragit® L & S). Optionally, the enteric coating contains a plasticiser. Examples of the plasticiser include, but are not limited to, triethyl citrate, triacetin, and diethyl phthalate.

The active compounds may also be in micro-encapsulated form, if appropriate, with one or more of the above-mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art such as water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan and mixtures thereof. Besides inert diluents, the oral compositions may also include adjuvants such as wetting agents, emulsifying and suspending agents, sweetening, flavouring and perfuming agents. Suspensions, in addition to the active compounds, may contain suspending agents such as ethoxylated isostearyl alcohols, polyoxyethylene sorbitol and sorbitan esters, microcrystalline cellulose, aluminium metahydroxide, bentonite, agar-agar, and tragacanth and mixtures thereof.

The products of the disclosure may be presented as solids in finely divided solid form, for example they may be micronised. Powders or finely divided solids may be encapsulated.

The active compound may be given as a single dose, in multiple doses or as a sustained release formulation.

According to a further aspect there is provided a parenteral formulation including a salt as described herein. The formulation may consist of the salt alone or it may contain additional components, in particular the salt may be in combination with a pharmaceutically acceptable diluent, excipient or carrier, for example a tonicity agent for the purpose of making the formulation substantially isotonic with the body of the subject to receive the formulation, e.g. with human plasma. The formulation may be in ready-to-use form or in a form requiring reconstitution prior to administration.

It is currently contemplated that, in the case of parenteral administration, for example i.v. administration, of salts of TRI 50c, the salts might for instance be administered in an amount of from 0.5 to 2.5 mg/Kg e.g. over a maximum period of 72 hours, calculated as TRI 50c. Other salts might be administered in equivalent molar amounts. The disclosure is not limited to administration in such quantities or regimens and includes dosages and regimens outside those described in the previous sentence.

Parenteral preparations can be administered by one or more routes, such as intravenous, subcutaneous, intradermal and infusion; a particular example is intravenous. A formulation disclosed herein may be administered using a syringe, injector, plunger for solid formulations, pump, or any other device recognized in the art for parenteral administration.

Liquid dosage forms for parenteral administration may include solutions, suspensions, liposome formulations, or emulsions in oily or aqueous vehicles. In addition to the active compounds, the liquid dosage forms may contain other compounds. Tonicity agents (for the purpose of making the formulations substantially isotonic with the subject's body, e.g. with human plasma) such as, for instance, sodium chloride, sodium sulfate, dextrose, mannitol and/or glycerol may be optionally added to the parenteral formulation. A pharmaceutically acceptable buffer may be added to control pH. Thickening or viscosity agents, for instance well known cellulose derivatives (e.g. methylcellulose, carboxymethylcellulose, hydroxyethylcellulose and hydroxypropylmethylcellulose), gelatin and/or acacia, may optionally be added to the parenteral formulation.

Solid dosage forms for parenteral administration may encompass solid and semi-solid forms and may include pellets, powders, granules, patches, and gels. In such solid dosage forms, the active compound is typically mixed with at least one inert, pharmaceutically acceptable excipient or carrier.

The disclosed salts may be presented as solids in finely divided solid form, for example they may be milled or micronised.

The formulations may also include antioxidants and/or preservatives. As antioxidants may be mentioned thiol derivatives (e.g. thioglycerol, cysteine, acetylcysteine, cystine, dithioerythreitol, dithiothreitol, glutathione), tocopherols, butylated hydroxyanisole, butylated hydroxytoluene, sulfurous acid salts (e.g. sodium sulfate, sodium bisulfite, acetone sodium bisulfite, sodium metabisulfite, sodium sulfite, sodium formaldehyde sulfoxylate, sodium thiosulfate) and nordihydroguaiareticacid. Suitable preservatives may for instance be phenol, chlorobutanol, benzylalcohol, methyl paraben, propyl paraben, benzalkonium chloride and cetylpyridinium chloride.

The parenteral formulations may be prepared as large volume parenterals (LVPs), e.g. larger than 100 ml, more particularly about 250 ml, of a liquid formulation of the active compound. Examples of LVPs are infusion bags. The parenteral formulations may alternatively be prepared as small volume parenterals (SVPs), e.g. about 100 ml or less of a liquid formulation of the active compound. Examples of SVPs are vials with solution, vials for reconstitution, prefilled syringes for injection and dual chamber syringe devices.

The formulations of the disclosure include those in which the salt is an alkali metal salt, for example a lithium, sodium or potassium salt, of which sodium salts may be mentioned as particular salts. Another class of formulations contains aminosugar salts of the disclosed boronic acids, for example N-methyl-D-glucamine salts. The salts mentioned in this paragraph may be administered as solutions in water, typically containing one or more additives, for example isotonicity agent(s) and/or antioxidant(s). A suitable way to store the salts is in solid form, for example as dry powder, and to make them up into solutions for administration prior to administration.

One class of formulations disclosed herein is intravenous formulations. For intravenously administered formulations, the active compound or compounds can be present at varying concentrations, with a carrier acceptable for parenteral preparations making up the remainder. Particularly, the carrier is water, particularly pyrogen free water, or is aqueous based. Particularly, the carrier for such parenteral preparations is an aqueous solution comprising a tonicity agent, for example a sodium chloride solution.

By “aqueous based” is meant that formulation comprises a solvent which consists of water or of water and water-miscible organic solvent or solvents; as well as containing a salt of disclosure in dissolved form, the solvent may have dissolved therein one or more other substances, for example an antioxidant and/or an isotonicity agent. As organic co-solvents may be mentioned those water-miscible solvents commonly used in the art, for example propyleneglycol, polyethyleneglycol 300, polyethyleneglycol 400 and ethanol. Preferably, organic co-solvents are only used in cases where the active agent is not sufficiently soluble in water for a therapeutically effective amount to be provided in a single dosage form. As previously indicated, the disclosure includes formulations of alkali metal salts of the disclosed boronic acids, e.g. TRI 50c, having a solvent which consists of water.

The solubility of the active compound in the present formulations may be such that the turbidity of the formulation is lower than 50 NTU, e.g. lower than 20 NTU such as lower than 10 NTU.

It is desirable that parenteral formulations are administered at or near physiological pH. It is believed that administration in a formulation at a high pH (i.e., greater than 8) or at a low pH (i.e., less than 5) is undesirable. In particular, it is contemplated that the formulations would be administered at a pH of between 6.0 and 7.0 such as a pH of 6.5.

In order to reduce the likelihood of forming oxidative degradation products, the parenteral formulation may be purged of air when being packaged. The parenteral formulation may be packaged in a sterile container, e.g. vial, as a solution, suspension, gel, emulsion, solid or a powder. Such formulations may be stored either in ready-to-use form or in a form requiring reconstitution prior to administration.

Parenteral formulations according to the disclosure may be packaged in containers. Containers may be chosen which are made of material which is non-reactive or substantially non-reactive with the parenteral formulation. Glass containers or plastics containers, e.g. plastics infusion bags, may be used. A concern of container systems is the protection they afford a solution against UV degradation. If desired, amber glass employing iron oxide or an opaque cover fitted over the container may afford the appropriate UV protection.

Plastics containers such as plastics infusion bags are advantageous in that they are relatively light weight and non-breakable and thus more easily stored. This is particularly the case for Large Volume parenterals.

The intravenous preparations may be prepared by combining the active compound or compounds with the carrier. After the formulation is mixed, it may be sterilized, for example using known methods. Once the formulation has been sterilized, it is ready to be administered or packaged, particularly in dark packaging (e.g. bottles or plastics packaging), for storage. It is envisaged, however, that the disclosed salts might not be stored in solution but as dry solids, particularly a finely divided form such as, for example, a lyophilisate, in order to prolong shelf life; this would of course apply to other parenteral formulations, not only intravenous ones.

The intravenous preparations may take the form of large volume parenterals or of small volume parenterals, as described above.

In a specific embodiment, the present disclosure is directed to products, particularly kits, for producing a single-dose administration unit. The products (kits) may each contain both a first container having the active compound (optionally combined with additives, for example anti-oxidant, preservative and, in some instances, tonicity agent) and a second container having the carrier/diluent (for example water, optionally containing one or more additives, for example tonicity agent). As examples of such products may be mentioned single and multi-chambered (e.g. dual-chamber) pre-filled syringes; exemplary pre-filled syringes are available from Vetter GmbH, Ravensburg, Germany. Such dual chamber syringes or binary syringes will have in one chamber a dry preparation including or consisting of the active compound and in another chamber a suitable carrier or diluent such as described herein. The two chambers are joined in such a way that the solid and the liquid mix to form the final solution.

One class of formulations disclosed herein comprises subcutaneous or intradermal formulations (for example formulations for injection) in which the active salt (or active agent combination) is formulated into a parenteral preparation that can be injected subcutaneously or intradermally. The formulation for administration will comprise the active salt and a liquid carrier.

The carrier utilized in a parenteral preparation that will be injected subcutaneously or intradermally may be an aqueous carrier (for example water, typically containing an additive e.g. an antioxidant and/or an isotonicity agent) or a nonaqueous carrier (again one or more additives may be incorporated). As a non-aqueous carrier for such parenteral preparations may be mentioned highly purified olive oil.

The active compound and the carrier are typically combined, for example in a mixer. After the formulation is mixed, it is preferably sterilized, such as with U.V. radiation. Once the formulation has been sterilized, it is ready to be injected or packaged for storage. It is envisaged, however, that the disclosed salts will not be stored in liquid formulation but as dry solids, in order to prolong shelf life.

For making subcutaneous implants, the active salt may suitably be formulated together with one or more polymers that are gradually eroded or degraded when in use, e.g. silicone polymers, ethylene vinylacetate, polyethylene or polypropylene.

Transdermal formulations may be prepared in the form of matrices or membranes, or as fluid or viscous formulations in oil or hydrogels or as a compressed powder pellet. For transdermal patches, an adhesive which is compatible with the skin may be included, such as polyacrylate, a silicone adhesive or polyisobutylene, as well as a foil made of, e.g., polyethylene, polypropylene, ethylene vinylacetate, polyvinylchloride, polyvinylidene chloride or polyester, and a removable protective foil made from, e.g., polyester or paper coated with silicone or a fluoropolymer. For the preparation of transdermal solutions or gels, water or organic solvents or mixtures thereof may be used. Transdermal gels may furthermore contain one or more suitable gelling agents or thickeners such as silicone, tragacanth, starch or starch derivatives, cellulose or cellulose derivatives or polyacrylic acids or derivatives thereof. Transdermal formulations may also suitably contain one or more substances that enhance absorption though the skin, such as bile salts or derivatives thereof and/or phospholipids. Transdermal formulations may be prepared according to a method disclosed in, e.g., B W Barry, “Dermatological Formulations, Percutaneous Absorption”, Marcel Dekker Inc., New York—Basel, 1983, or Y W Chien, “Transdermal Controlled Systemic Medications”, Marcel Dekker Inc., New York—Basel, 1987.

It will be understood from the aforegoing that there are provided pharmaceutical products comprising an alkali metal salt, particularly sodium salt, of a boronic acid of Formula (I) in dry fine particle form, suitable for reconstitution into an aqueous ready-to-use parenteral formulation. The alkali metal salt is suitably an acid salt. The alkali metal salt may be in a small volume parenteral unit dosage form. The alkali metal salt may be presented in a form, e.g. dry powder form, suitable for reconstituting as a large volume parenteral. One example is a sodium salt of a boronic acid of Formula (I), particularly TRI 50c, in dry powder form for reconstitution as a liquid intravenous formulation (solution) containing a tonicity agent, particularly sodium chloride. The dry powder form of a salt used in parenteral formulation may be a lyophilisate. The reconstituted solution may be administered by injection or infusion.

Boropeptide Synthesis

The synthesis of boropeptides, including, for example, Cbz-D-Phe-Pro-BoroMpg-OPinacol is familiar to those skilled in the art and described in the prior art mentioned above, including Claeson et al (U.S. Pat. No. 5,574,014 and others) and Kakkar et al (WO 92/07869 and family members including U.S. Pat. No. 5,648,338). It is described also by Elgendy et al Adv. Exp. Med. Biol. (USA) 1993, 340, 173-178; Claeson,G. et al Biochem.J. 1993, 290, 309-312; Deadman et al J. Enzyme Inhibition 1995, 9, 29-41, and by Deadman et al J. Med. Chem. 1995, 38, 1511-1522.

Stereoselective synthesis with S or R configuration at the chiral B-terminal carbon may be conducted using established methodology (Elgendy et al Tetrahedron. Lett. 1992, 33, 4209-4212; WO 92/07869 and family members including U.S. Pat. No. 5,648,338) using (+) or (−)-pinanediol as the chiral director (Matteson et al J. Am. Chem. Soc. 1986, 108, 810-819; Matteson et al Organometallics. 1984, 3, 1284-1288). Another approach is to resolve the requisite aminoboronate intermediate (e.g. Mpg-BOPinacol) to selectively obtain the desired (R)-isomer and couple it to the dipeptide moiety (e.g. Cbz-(R)-Phe-(S)-Pro, which is the same as Cbz-D-Phe-L-Pro) which will form the remainder of the molecule.

### EXAMPLES

Abbreviations

The following abbreviations are used in the examples:

| | |
|---|---|
| area | peak area |
| AU | absorption unit |
| b | intercept on y-axis of the calibration curve |
| conc | concentration |
| DMSO | dimethylsulfoxide |
| Inj | injection |
| ID | inner diameter |
| KLP | calibration solution |
| KRB | Krebs Ringer buffer |
| L | length |
| LOD | limit of detection |
| LOQ | limit of quantification |
| m | slope of the calibration curve |
| MS | mass spectrometry |
| MW | molecular weight |
| M/z | Mass/charge rate |
| n | number of analysed samples |
| pa | pro analysis |
| PDA | Photo diode array |
| r | correlation coefficient |
| Rₛ | resolution of neighboring peaks |
| RSD | relative standard deviation |
| Ret Time | retention time |
| SD | standard deviation |
| SL | stock solution |
| temp | temperature |
| TK | freezer |
| μAU's | area under the peak |
| | (taken from Millenium³² software) |

In the Examples, all the values are within experimental error.

Examples 1 to 3 describe performance of the following reaction scheme and conversion of the resultant TRI 50c to sodium and calcium salts thereof:

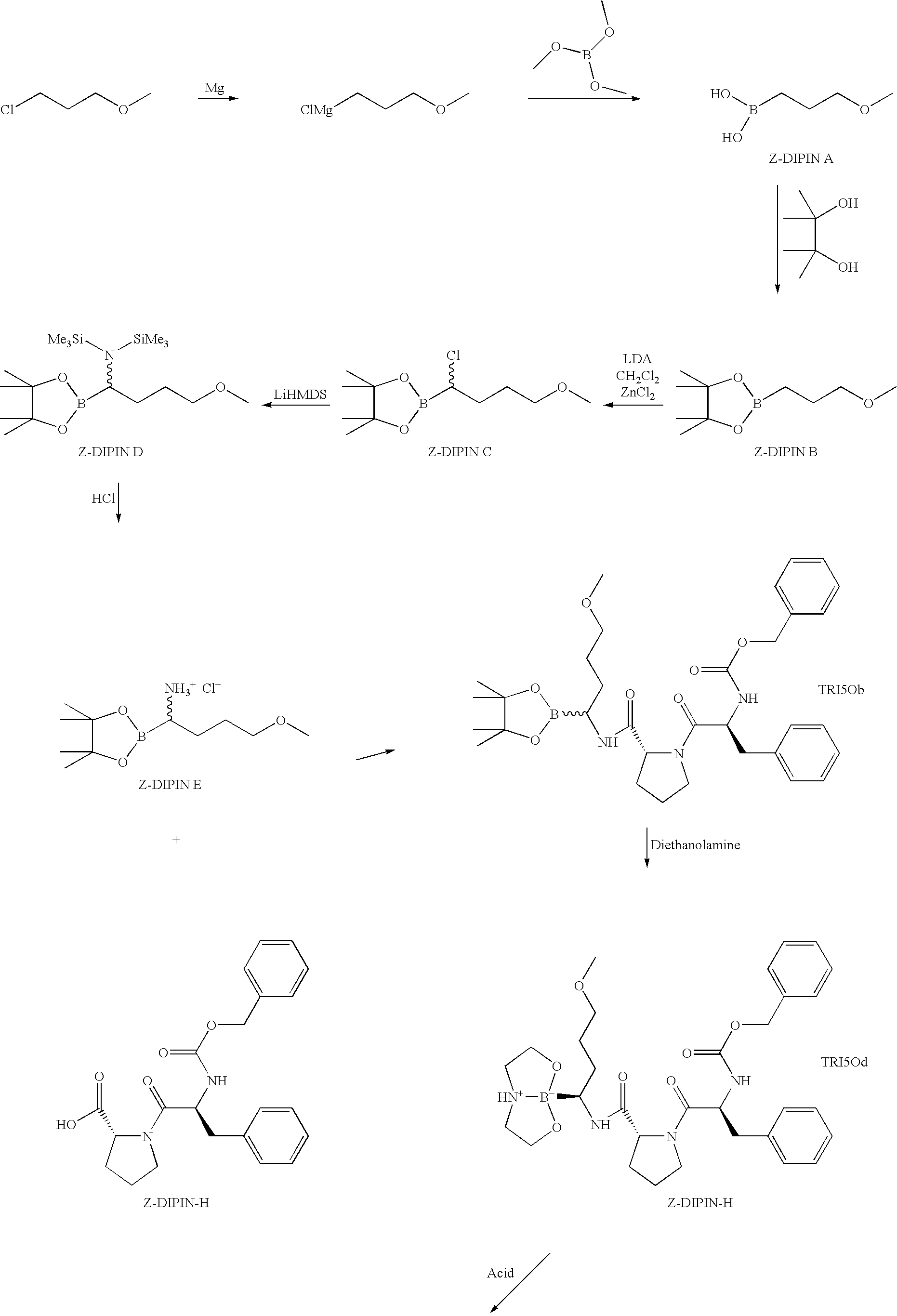

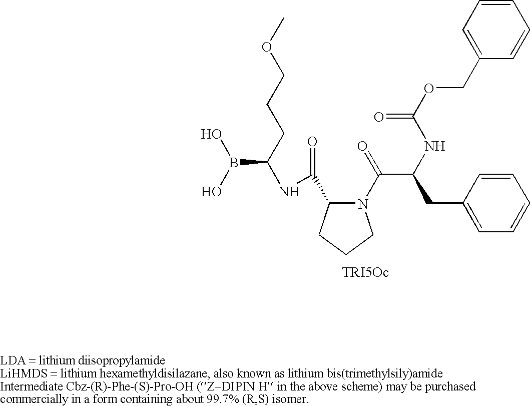

### Example 1

### Synthesis of TRI 50D

Apparatus

Throughout the following procedures, standard laboratory glassware and, where appropriate, specialised apparatus for handling and transferring of air sensitive reagents are used.

All glassware is heated at 140-160° C. for at least 4 hours before use and then cooled either in a desiccator or by assembling hot and purging with a stream of dry nitrogen.

Dryness

In the drying procedures of the following examples, products are tested for dryness (including dryness in terms of organic solvent) by observing weight loss on drying. The following procedure was followed to determine loss on drying: a sample was placed in a vacuum drier and dried at 40° C. at 100 mbar for 2 hours. Products are considered dry when the decrease in weight upon drying is less than 0.5% of the total weight of the starting material.

Solvents

The organic solvents used in the procedures of Examples 1, 2 and 3 are all dry. Suitably, they are dried over sodium wire before use.

Step 1: Z-DIPIN B

Procedure A

17.8 g (732.5 mmole) magnesium turnings, 0.1 g (0.4 mmole) iodine and 127 ml dry tetrahydrofuran are charged and heated to reflux. Then 15 ml of a solution of 66 g (608 mmole) 1-chloro-3-methoxypropane in 185 ml dry tetrahydrofuran are added and stirred under reflux until the vigorous reaction starts. After the initial exotherm ceases, the solution of 1-chloro-3-methoxypropane is added slowly to maintain gentle reflux until all the magnesium is consumed. After the reaction is finished, the reaction mixture is cooled to ambient temperature and slowly added to a solution of 64.4 g (620 mmole) trimethylborate in 95 ml dry tetrahydrofuran; the latter solution is cooled to below 0° C. and, if it warms up during the course of the reaction, the reaction mixture must be added to it sufficiently slowly to maintain the temperature of this solution below 65° C. Upon complete addition, the reaction mixture is allowed to warm to about 0° C. and stirred for another 60 minutes. Then a solution of 22.4 ml sulfuric acid in 400 ml water is added slowly so as to maintain the temperature below 20° C. The layers are allowed to settle and the phases are separated. The aqueous layer is rewashed three times with 200 ml tert.-butylmethylether. The combined organic layers are allowed to settle and additional water separated from this solution is removed. The organic layer is dried over magnesium sulfate, filtered and evaporated to dryness. The evaporation residue is filtered from the precipitated solid and the filtrate dissolved in 175 ml toluene. 34.8 g (292 mmole) pinacol is charged to the solution followed by stirring at ambient temperature for not less than 10 hours. The solution is evaporated to dryness, dissolved in 475 ml n-heptane and washed three times with 290 ml saturated aqueous solution of sodium hydrogen carbonate. The n-heptane solution is evaporated to dryness and the evaporation residue distilled and the fraction with Bp 40-50° C. at 0.1-0.5 mbar recovered.

Boiling point: 40-50° C./0.1-0.5 mbar

Yield: 40.9 g (70%) Z-DIPIN B (oil)

Procedure B

17.8 g (732.5 mmole) magnesium turnings, 0.1 g (0.4 mmole) iodine and 127 ml dry tetrahydrofuran are charged and heated to reflux. Then 15 ml of a solution of 66 g (608 mmole) 1-chloro-3-methoxypropane in 185 ml dry tetrahydrofuran are added and stirred under reflux until the vigorous reaction starts. After the initial exotherm ceases, the solution of 1-chloro-3-methoxypropane is added slowly to maintain gentle reflux. After the reaction is finished, the reaction mixture is cooled to ambient temperature and slowly added to a solution of 64.4 g (620 mmole) trimethylborate in 95 ml dry tetrahydrofuran, maintaining the temperature of this solution below minus 65° C. Upon complete addition, the reaction mixture is allowed to warm to about 0° C. and stirred for another 60 minutes. Then a solution of 22.4 ml sulfuric acid in 400 ml water is added slowly so as to maintain the temperature below 20° C. The organic solvent is removed by distillation under vacuum. 300 ml n-heptane is charged to the aqueous solution of the evaporation residue followed by addition of 34.8 g (292 mmole) pinacol. The two-phase-mixture is stirred at ambient temperature for not less than 2 hours. After allowing the layers to settle, the aqueous phase is separated. 300 ml n-heptane is charged to the aqueous solution and the two-phase-mixture is stirred at ambient temperature for not less than 2 hours. After allowing the layers to settle, the aqueous phase is separated. The organic layers are combined and washed once with 200 ml water, followed by 200 ml saturated sodium hydrogen carbonate solution and two further washes with 200 ml water each. The n-heptane solution is evaporated to dryness and the evaporation residue distilled and the fraction with Bp 40-50° C. at 0.1-0.5 mbar recovered.

Boiling point: 40-50° C./0.1-0.5 mbar

Yield: 40.9 g (70-85%) Z-DIPIN B (oil)

Step 2: Z-DIPIN C

16.6 g (164 mmole) diisopropylamine and 220 ml tetrahydrofuran are charged and cooled to −30 to −40° C. To this solution 41.8 g (163 mmole) n-butyl lithium, 25% in n-heptane is added, followed by stirring at 0 to −5° C. for one hour. This freshly prepared solution of lithium diisopropylamide is cooled to −30° C. and then added to a solution of 27.9 g (139 mmole) Z-DIPIN B in 120 ml tetrahydrofuran and 35.5 g (418 mmole) dichloromethane at a temperature between −60 and −75° C. The solution is stirred at that temperature for half an hour followed by addition of 480 ml (240 mmole) 0.5N anhydrous Zinc(II)-chloride in tetrahydrofuran or 32.5 g (240 mmole) anhydrous solid Zinc(II)-chloride. After stirring at −65° C. for one hour, the reaction mixture is allowed to warm to ambient temperature and stirred for another 16-18 hours. The reaction mixture is evaporated to dryness (i.e. until solvent is removed) and followed by addition of 385 ml n-heptane. The reaction mixture is washed with 150 ml 5% sulfuric acid, with 190 ml saturated sodium hydrogen carbonate solution, and 180 ml saturated sodium chloride solution. The organic layer is dried over magnesium sulfate, filtered and evaporated to dryness (i.e. until solvent is removed). The oily residue is transferred into the next step without further purification.

Yield: 19 g (55%) Z-DIPIN C

Step 3: Z-DIPIN D

To a solution of 23.8 g (148 mmole) hexamethyidisilazane in 400 ml tetrahydrofuran at −15° C. is added 34.7 g (136 mmole) n-butyl lithium, 25% in n-heptane and stirred for one hour. The solution is cooled to −55° C. followed by the addition of 30.6 g (123 mmole) Z-DIPIN C dissolved in 290 ml tetrahydrofuran and 35 ml tetrahydrofuran to this freshly prepared solution of LiHMDS. The solution is allowed to warm to ambient temperature and stirred for 12 hours. The reaction mixture is evaporated to dryness, the evaporation residue dissolved in 174 ml n-heptane, washed with 170 ml water and 75 ml saturated sodium chloride solution. The organic phase is dried over magnesium sulfate, filtered and evaporated to complete dryness (i.e. until solvent is removed). The oily residue is dissolved in 100 g n-heptane. This solution is carried over into the next step without further purification.

Yield: 32.2 g (70%) Z-DIPIN D

Step 4: Z-DIPIN (TRI50b, crude)

A solution of 26.6 g (71 mmole) Z-DIPIN D in 82.6 g n-heptane is diluted with 60 ml n-heptane and cooled to −60° C. followed by introduction of 10.5 g (285 mmole) hydrogen chloride. The reaction mixture is subsequently evacuated and flushed with nitrogen, while the temperature is increased in increments of about 20° C. to ambient temperature. The solvent is removed from the oily precipitate and replaced several times by 60 ml fresh n-heptane. The oily residue is dissolved in 60 ml tetrahydrofuran (Solution A).

To a different flask 130 ml tetrahydrofuran, 24.5 g (61.5 mmole) Z-D-Phe-Pro-OH and 6.22 g (61.5 mmole) N-methylmorpholine are charged and cooled to −20° C. To this solution a solution of 8.4 g (61.5 mmole) isobutylchloroformate in 20 ml tetrahydrofuran is added and stirred for 30 minutes, followed by addition of Solution A at −25° C. Upon complete addition, up to 16 ml (115 mmole) triethylamine is added to adjust the pH to 9-10, measured using a pH stick. The reaction mixture is allowed to warm to ambient temperature and stirred for 3 hours, still under nitrogen. The solvent is evaporated to dryness and the evaporation residue dissolved in 340 ml tert.-butylmethylether (t-BME). The solution of Z-DIPIN in t-BME is washed twice with 175 ml 1.5% hydrochloric acid. The combined acidic washes are given a rewash with 175 ml t-BME. The combined organic layers are washed with 175 ml water, with 175 ml saturated sodium hydrogen carbonate solution, with 175 ml 25% sodium chloride solution, dried over magnesium sulfate and filtered. This solution is carried over into the next step without further purification.

Yield: 29.9 g (80%) Z-DIPIN

Step 5: TRI50d

The solution of Z-DIPIN in t-BME (containing 7.0 g (11.5 mmole) (R,S,R) TRI50b, calculated based on HPLC results of Z-DIPIN) is evaporated to dryness and the evaporation residue dissolved in 80 ml diethylether. 1.51 g (14.4 mmole) diethanolamine is added and the mixture heated at reflux for at least 10 hours, during which process the product precipitates. The suspension is cooled to 5-10° C., filtered and the filter residue washed with diethylether.

To improve chiral and chemical purity the wet filter cake (7 g) is dissolved in 7 ml dichloromethane, cooled to 0-5° C. and the product precipitated by addition of 42 ml diethylether and filtered. The isolated wet product is dried at 35° C. in vacuum or at least 4 hours, until day.

Yield: 5.5 g (80%) Tri50d

Melting Point: 140-145° C.

### Example 2

### Preparation of Sodium Salt of TRI50C (TGN 255)

1.5 kg (2.5 mole) TRI50d is dissolved in 10.5 L dichloromethane. 11 L 2% hydrochloric acid is added and the mixture is stirred for at most 30 minutes (optimally about 20 minutes) at room temperature. A precipitate forms in the organic phase. After stirring, the layers are allowed to settle and separated. The aqueous layer is rewashed twice with 2.2 L dichloromethane. The combined organic layers are washed with a solution of 625 g ammonium chloride in 2.25 L water. (The ammonium chloride buffers the pH of the aqueous extractions to be within a range of from about pH 1-2 to about pH 4-5, as strongly acidic conditions might cleave peptide bonds). The organic phase is dried over magnesium sulfate, filtered and the filtrate evaporated to dryness. An assay of the free boronic acid is performed (by the RP HPLC method of Example 5 for at most 30 mins (optionally about 20 min) at room temperature) and the amounts of the solvents and base for conversion of the acid to the salt are calculated. If 2.5 mol of the free acid is obtained, the evaporation residue is dissolved in 5 L acetonitrile followed by addition of a solution of 100 g (2.5 mole) sodium hydroxide as a 5% solution in 2.2 L water. The solution is stirred for two hours at ambient temperature (e.g. 15-30° C., optimally room temperature) and then evaporated in vacuum (of ca. 10 mmHg) at a temperature not exceeding 35° C. The evaporation residue is repeatedly dissolved in 3.5 L fresh acetonitrile and evaporated to dryness to remove traces of water. If the evaporation residue is dry, it is dissolved in 3 L acetonitrile (or alternatively in 6 L THF) and slowly added to a mixture of 32 L n-heptane and 32 L diethylether. The addition is performed slowly enough to avoid lumping or sticking of the product and is carried out over a period of not less than 30 minutes. The precipitated product is filtered off, washed with n-heptane and dried under vacuum at a temperature initially of about 10° C. and then increasing to a limit of about 35° C., until dry.

Yield: 1.0 kg (70%) Tri50c sodium salt.

### Example 3

### Preparation of Calcium Salt of TRI50C (TGN 167)

1.5 kg (2.5 mole) TRI50d is dissolved in 10.5 L dichloromethane. 11 L 2% hydrochloric acid is added and the mixture is stirred for at most 30 minutes (optimally about 20 minutes) at room temperature. After stirring the layers are allowed to settle and separated. The aqueous layer is rewashed twice with 2.2 L dichloromethane. The combined organic layers are washed with a solution of 625 g ammonium chloride in 2.25 L water. The organic phase is dried over magnesium sulfate, filtered and the filtrate evaporated to dryness. An assay of the free boronic acid is performed and the amounts of the solvents and base for conversion of the acid to the salt are calculated. If 2.5 mol of the free acid is obtained, the evaporation residue is dissolved in 5 L acetonitrile followed by addition of a suspension of 93 g (1.25 mole) calcium hydroxide in 1 L water. The solution is stirred for two hours at ambient temperature (e.g. 15-30° C., optimally room temperature) and then evaporated under vacuum (of ca. 10 mmHg) at a temperature initially of about 10° C. and then increasing to a limit of about 35° C. The evaporation residue is repeatedly dissolved in 3.5 L fresh acetonitrile and evaporated to dryness to remove traces of water. If the evaporation residue is dry, it is dissolved in 6 L tetrahydrofuran and slowly added to a mixture of 32 L n-heptane and 32 L diethylether. The addition is performed slowly enough to avoid lumping or sticking of the product and is carried out over a period of not less than 30 minutes. The precipitated product is filtered off, washed with n-heptane and dried under vacuum (of ca. 10 mmHg) at a temperature below 35° C. until dry.

Yield: 0.98 kg (70%) Tri50c calcium salt.

### Example 4

### Residual n-Heptane of TRI 50C Calcium Salt

Salt prepared following the methods of Examples 1 and 3 was tested by headspace gas chromatography. Data are shown below:

Residual solvents: Headspace gas chromatography

| | |
|---|---|
| GC Parameter: | |
| Column: | DB-wax, 30 m, 0.32 mm ID, 5 μ |
| Carrier Gas: | Helium 5.0, 80 kPas |
| Detector: | FID, 220° C. |
| Injector Temp: | 150° C. |
| Operating Conditions: | 35° C./7 min; 10° C./min up to 80° C./2 min; |
| | 40° C. up to 180° C./2 min |
| Injection volume: | 1 ml |
| Split: | On |
| Headspace Parameter: | |
| Oven temperature: | 70° C. |
| Needle temperature: | 90° C. |
| Transfer temperature: | 100° C. |
| Other parameters: | temper time: 15 min, GC-cycle time: 28 min, |
| | injection time: 0.03 min, duration: 0.4 min |

Calibration Standards: sample weight/dilution

| | | | concentration | area (average, |
|---|---|---|---|---|
| standard | weight (mg) | volume (ml) | (mg/ml) | n = 3) |
| n-heptane | 103.12 | 100 | 1.0312 | 2757.74756 |
| | | | concentration | |
| sample no. | weight (mg) | volume (ml) | (mg/ml) | |
| 1 | 100.84 | 5 | 20.17 | |
| 2 | 99.12 | 5 | 19.82 | |
| 3 | 100.03 | 5 | 20.01 | |

n-heptane

| sample | concentration (mg/ml) | content % |
|---|---|---|
| 1 | 0.0010 | 0.0048 |
| 2 | 0.0009 | 0.0044 |
| 3 | 0.0010 | 0.0050 |
| | 0.00095 | 0.005 |

### Example 5

### HPLC Chromatograms

TRI 50c monosodium salt made by the method of Examples 1 & 2 and TRI 50c hemicalcium salt made by the method of Examples 1 and 3 were analysed by HPLC chromatography.

1. Method

1.1 Equipment and Software

| | |
|---|---|
| Autosampler | Waters Alliance 2795 |
| Pump | Waters Alliance 2795 |
| Column oven | Waters Alliance 2795 |
| Detection | Waters 2996 diode array, MS-ZQ single quad |
| Software version | Waters Millennium 4.0 |

1.2 Stationary Phase

| | |
|---|---|
| Analytical Column ID | S-71 |
| Material | XTerra ™ MS C₁₈, 5 μm |
| Supplier | Waters, Eschborn, Germany |
| Dimension | 150 mm × 2.1 mm (length, ID) |
| Pre-column ID | no pre-column |

Xterra MS C₁₈, 5 μm is a column packing material supplied by Waters Corporation, 34 Maple Street, Milford, Mass. 01757, US and local offices, as in years 2002/2003. It comprises hybrid organic/inorganic particles, consisting of spherical particles of 5 μm size, 125 Å pore size and 15.5% carbon load.

1.3 Mobile Phase

| | |
|---|---|
| Aqueous phase: | A: H₂O + 0.1% HCOOH |
| Organic phase: | C: ACN |

H₂O = H₂O by Ultra Clear water purification system
ACN = gradient grade acetonitrile

Gradient Conditions

| time | | | flow | gradient |
|---|---|---|---|---|
| [min] | A % | C % | [mL/min] | shape |
| 0.0 | 90.0 | 10.0 | 0.5 | |
| 27.00 | 10.0 | 90.0 | 0.5 | linear |
| 27.10 | 90.0 | 10.0 | 0.5 | linear |
| 30.00 | 90.0 | 10.0 | 0.5 | linear |

1.4 Instrumental Parameters

| | |
|---|---|
| Flow | 0.5 mL-min⁻¹ |
| Temperature | 40 ± 5° C. |
| HPLC control | Waters Millennium Release 4.0 |
| Calculation | Waters Millenium 4.0 |

2. Parameters

2.1 Wavelength/Retention Time/Response Factors

TABLE
retention and detection parameter (k′ F: 0.5 ml/min, t0 = 0.9 mL/min)

| | | | | response | Reciprocal |
|---|---|---|---|---|---|
| | RetTime | λ | | factor | Response |
| Substance | [min] | [nm] | m/z | [area/μg] | factor |
| TRI 50c | 11.68 | 258 | 508.33 | 660 | 1 |
| Benzyl alcohol | 3.862 | 258 | n.d. | 1960 | 0.337 |
| Benzaldehyde | 6.13 | 258 | n.d. | 79939 | 0.0083 |
| Benzoic acid | 5.52 | 258 | n.d. | 5967 | 0.111 |
| Impurity I | 11.18 | 258 | 396.17 | 886 | 0.745 |
| Impurity II | 13.39 | 258 | 482.22 | 552 | 1.196 |

2.2 Linearity

Linearity Range 4000-10 μg/mL (detection UV 258 nm)

TABLE
Linearity data UV 258 nm

| calibration | area | target conc. | conc. found¹ |
|---|---|---|---|
| solution | [μAU's] | [μg/mL] | [μg/mL] |
| Tri 50c | 5353 | 10 | 20.44 |
| Tri 50c | 5301 | 10 | 20.37 |
| Tri 50c | 65809 | 100 | 113.35 |
| Tri 50c | 66365 | 100 | 114.17 |
| Tri 50c | 172019 | 250 | 270.43 |
| Tri 50c | 162587 | 250 | 256.48 |
| Tri 50c | 339503 | 500 | 518.13 |
| Tri 50c | 326912 | 500 | 499.51 |
| Tri 50c | 659257 | 1000 | 991.02 |
| Tri 50c | 647495 | 1000 | 973.63 |
| Tri 50c | 1322371 | 2000 | 1971.72 |
| Tri 50c | 1305196 | 2000 | 1946.32 |
| Tri 50c | 2724410 | 4000 | 4045.24 |

¹recalculated with linear equation

Linear Equation Parameters:
Y=6.75e+002 X−8.45e+003
r=0.99975
r²=0.99950
Linearity Range 10-0.10 μg/mL (detection SIR m/z 508.33)

Table: Linearity data SIR 508.33

| calibration | mean area | target conc. | conc. found¹ |
|---|---|---|---|
| solution | [μAU's] | [μg/mL] | [μg/mL] |
| Tri 50c | 2188860 | 0.01 | 0.022 |
| Tri 50c | 2702839 | 0.01 | 0.045 |
| Tri 50c | 3817226 | 0.1 | 0.094 |
| Tri 50c | 3833799 | 0.1 | 0.095 |
| Tri 50c | 23153550 | 1 | 0.947 |
| Tri 50c | 24646892 | 1 | 1.013 |
| Tri 50c | 223007852 | 10 | 9.765 |
| Tri 50c | 233753043 | 10 | 10.239 |

¹recalculated with linear equation

Equation Parameter
Y=2.27e+007 X+1.69e+006
r=0.99958
r²=0.99916
2.3 Quantitation Limit

The quantitation limit was determined using the signal to noise ratio criterion S/N>19,
UV 258 nm: 10 μg/mL
M/z 508.3: 0.1 μg/mL

2.4 Precision

| | Target concentration | | Amount | Retention time |
|---|---|---|---|---|
| Injection | [μg/mL] | Area | [μg/mL] | [min] |
| 1 | 250 | 165805 | 261.24 | 11.690 |
| 2 | 250 | 168644 | 265.44 | 11.662 |
| 3 | 250 | 167858 | 264.27 | 11.686 |
| 4 | 250 | 166947 | 262.93 | 11.692 |
| 5 | 250 | 166925 | 262.89 | 11.679 |
| 6 | 250 | 166294 | 261.96 | 11.696 |
| Mean | | 167079 | 263.12 | 11.684 |
| Std. Dev. | | 1033 | 1.528 | 0.01 |
| % RSD | | 0.6 | 0.6 | 0.1 |

2.5 Robustness

TABLE
robustness data; Standard 250 μg/mL aqueous solution
(containing <1% ACN)

| calibration | temp./time | | recovery |
|---|---|---|---|
| solution | [° C./h] | area [μAU's] | [%] |
| 250 μg/mL Tri50c | — | 172020 | — |
| 250 μg/mL Tri50c | 4° C. 16 h | 166294 | 96.67 |
| 2.5 μg/mL TRI50c | — | 88034891 | — |
| 2.5 μg/mL TRI50c | 37° C. 4 h | 88833175 | 100.9 |

References

1. ICH HARMONISED TRIPARTITE GUIDELINE. TEXT ON VALIDATION OF ANALYTICAL PROCEDURES Recommended for Adoption at Step 4 of the ICH Process on 27 Oct. 1994 by the ICH Steering Committee

2. FDA Reviewer Guidance. Validation of chromatographic methods. Center for Drug Evaluation and Research. November 1994

3. USP 23. <621>Chromatography

4. L. Huber. Validation of analytical Methods. LC-GC International February 1998

5. Handbuch Validierung in der Analytik. Dr. Stavros Kromidas (Ed.) Wiley-VCH Verlag. 2000. ISBN 3-527-29811-8

3. Results

3.1 Sample Name: TRI 50c Monosodium Salt

Injection Volume: 10 μL

| | Ret Time | | Area | Peak Height |
|---|---|---|---|---|
| Name | (Min) | Area % | [μAU's] | μAU |
| TRI 50c | 12.136 | 100.0000 | 604.27228 | 32.05369 |

3.2 Sample Name: TRI 50c Hemicalcium Salt

Injection Volume: 10 μL

| | Ret Time | | Area | Peak Height |
|---|---|---|---|---|
| Name | (Min) | Area % | [μAU's] | μAU |
| TRI 50c | 12.126 | 100.0000 | 597.11279 | 32.29640 |

The disclosed methods have been used to obtain salts substantially free of C—B bond degradation products, in particular salts containing no such products in an amount detectable by HPLC, specifically the method of Example 5. The disclosed methods have been used to obtain salts substantially free of Impurity I, in particular containing no Impurity I in an amount detectable by HPLC, specifically by the method of Example 5. The disclosed methods have been used to obtain salts substantially free of Impurity IV, in particular containing no Impurity IV in an amount detectable by HPLC, specifically by the method of Example 5.

### Example 6

### Determination of Diastereomeric Excess

TRI 50b, crude, contains three chiral centres. Two of them are fixed by the use of enantiomerically pure amino acids ((R)-Phe and (S)-Pro). The third one is formed during the synthesis. The favoured epimer is the desired TRI 50b, Isomer I (R,S,R-TRI 50b). Both epimers of TRI 50b are clearly baseline separated by the HPLC method, thus allowing determination of the diasteromeric excess (de) of TRI 50b.

TRI 50d is not stable under the conditions applied for HPLC purity determination, but decomposes rapidly on sample preparation to TRI 50c, so that TRI 50d and TRI 50c show the same HPLC traces.

The two isomers of TRI 50c are not baseline separated in HPLC, but both isomers are clearly visible. This becomes obvious, when TRI 50b, crude (mixture of both isomers) is converted with phenylboronic acid to TRI 50c, crude. Both isomers of TRI 50c are observed in HPLC nearly at the same relation as before in TRI 50b, crude.

Upon synthesis of TRI 50d from TRI 50b, crude, only one diastereoisomer is precipitated. In this case HPLC shows only one peak for TRI 50c, where a very small fronting is observed. Precipitation from dichloromethane/diethylether removes the fronting efficiently. The level of removal of Isomer II cannot be quantified by this HPLC method. Therefore samples before reprecipitation and after one and two reprecipitations were esterified with pinacol and the resulting samples of TRI 50b analysed by HPLC. Thus a de of 95.4% was determined for the crude sample. The reprecipitated sample resulted in a de of 99.0% and finally the sample that was reprecipitated twice showed a de of 99.5%.

These results clearly show the preferred precipitation of Isomer I, whereas Isomer II remains in solution.

### Comparative Example A

Previous Conversion of TRI 50b to TRI 50c

1. Approximately 300 g of TRI 50b isomer (obtained by the HPLC purification of racemic TRI 50b) were dissolved in approximately 2.5 L diethylether.

2. Approximately 54 ml diethanolamine were added (1:1 stoichiometry with total TRI 50b content), and the mixture was refluxed at 40° C.

3. The precipitated product was removed, washed several times with diethylether and dried.

4. The dry product was dissolved in CHCl₃. Hydrochloric acid (ph 1) was added and the mixture was stirred approximately 1 h at room temperature.

5. The organic layer was removed and washed with NH₄Cl solution.

6. The organic solvent was distilled off and the residual solid product was dried.

Typical yield: Approximately 230 g.

Previous Preparation of Sodium Salt of TRI 50c

Cbz-Phe-Pro-BoroMpg-OH obtained by the previous method (20.00 g, 38.1 mM) is dissolved in acetonitrile (200 ml) with stirring at room temperature. To this solution is added NaOH as a 0.2M solution in distilled water (190 ml). The resultant clear solution is stirred for 2 hours at room temperature and then evacuated to dryness under vacuum with its temperature not exceeding 37° C. The resultant oil/tacky liquid is redissolved in 500 ml distilled water with light warming for about 15-20 minutes. The solution is filtered through filter paper and evacuated to dryness, again with the temperature of the solution not exceeding 37° C. The resultant product is dried under vacuum overnight to normally yield a white brittle solid. The product may be present as an oil or tacky solid due to residual water, in which case it is dissolved in ethyl acetate and evacuated to dryness to produce the product as a white solid.

The salt was then dried under vacuum over silica to constant weight (72 h). Further disclosed herein is the subject matter of the following paragraphs:

1. A process for separating diastereomers of a boronic acid of formula (I): 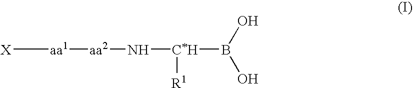
where:
X is H (to form NH₂) or an amino-protecting group;
aa¹ is an amino acid of (R) configuration selected from Phe, Dpa and wholly or partially hydrogenated analogues thereof;
aa² is an imino acid of (S) configuration having from 4 to 6 ring members;
R¹ is a group of the formula —(CH₂)₅-Z, where s is 2, 3 or 4 and Z is —OH, —OMe, —OEt or halogen (F, Cl, Br or I),
and where C* is a chiral centre,
   the process comprising:
combining in diethylether solution (A) a boronic species selected from the boronic acid (I) and its esters, the boronic species including molecules having a chiral centre C* of (R) configuration and molecules having a chiral centre C* of (S) configuration, and (B) diethanolamine, the diethanolamine being in an amount of about 1.25±0.1 equivalents based on the boronic species in which the chiral centre C* is of (R) configuration;
causing or allowing the boronic species and the diethanolamine to react until a precipitate forms; and
recovering the precipitate.

2. A process of paragraph 1 in which the diethanolamine is in an amount of from 1.2 to 1.3 equivalents based on the boronic species in which chiral centre C* is of (R) configuration.

3. A process of paragraph 2 in which the diethanolamine is in an amount of about 1.25 equivalents based on the boronic species.

4. A process of any of paragraphs 1 to 3 in which the alcohol is a diol.

5. A process of paragraph 4 in which the diol is not sterically hindered.

6. A process of paragraph 5 in which the diol is pinacol, neopentylglycol, 1,2-ethanediol, 1,2-propanediol, 1,3-propanediol, 2,3-butanediol, 1,2-diisopropylethanediol, or 5,6-decanediol.

7. A process of paragraph 6 in which the diol is pinacol.

8. A process of any of paragraphs 1 to 7 in which the boronic species and the diethanolamine are caused to react by heating the mixture to an elevated temperature.

9. A process of paragraph 8 in which the mixture is refluxed.

10. A process of paragraph 9 in which the mixture is refluxed for at least 10 hours.

11. A process of any of paragraphs 1 to 10 in which the precipitate is recovered by filtration.

12. A process of any of paragraphs 1 to 11 in which the recovered precipitate is washed with diethylether.

13. A process of any of paragraphs 1 to 12 in which the recovered precipitate, after washing in the event that the precipitate is washed, is dissolved in a solvent selected from CH₂Cl₂ and CHCl₃ and reprecipitated by combining the resulting solution with diethylether.

14. A process of paragraph 13 in which the solvent is CH₂Cl₂.

15. A process of any of paragraphs 1 to 14 in which aa¹ is selected from (R)-Dpa, (R)-Phe, (R)-Dcha and (R)-Cha.

16. A process of paragraph 15 in which aa¹ is (R)-Phe or (R)-Dpa.

17. A process of paragraph 15 in which aa¹ is (R)-Phe. (IV),

18. A process of any of paragraphs 1 to 17 in which aa² is a residue of an imino acid of formula (IV) 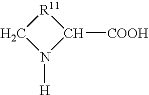
where R¹¹ is —CH₂—, —CH₂—CH₂—, —S—CH₂—, —S—C(CH₃)₂— or —CH₂—CH₂—CH₂—, which group, when the ring is 5- or 6-membered, is optionally substituted at one or more —CH₂— groups by from 1 to 3 C₁-C₃ alkyl groups.

19. A process of paragraph 18 in which aa² is (S)-proline.

20. A process of any of paragraphs 1 to 14, in which aa¹-aa² is (R)-Phe-(S)-Pro.

21. A process of any of paragraphs 1 to 20 in which R¹ is 2-bromoethyl, 2-chloroethyl, 2-methoxyethyl, 3-bromopropyl, 3-chloropropyl or 3-methoxypropyl.

22. A process of any of paragraphs 1 to 20 in which R¹ is 3-methoxypropyl.

23. A process of any of paragraphs 1 to 22 where X is R⁶—(CH₂)ₚ—C(O)—, R⁶—(CH₂)ₚ—S(O)₂—, R⁶—(CH₂)ₚ—NH—C(O)— or R⁶—(CH₂)ₚ—O—C(O)— in which p is 0, 1, 2, 3, 4, 5 or 6 and R⁶ is H or a 5 to 13-membered cyclic group optionally substituted by 1, 2 or 3 substituents selected from halogen, amino, nitro, hydroxy, a C₅-C₆ cyclic group, C₁-C₄ alkyl and C₁-C₄ alkyl containing, and/or linked to the cyclic group through, an in-chain O, the aforesaid alkyl groups optionally being substituted by a substituent selected from halogen, amino, nitro, hydroxy and a C₅-C₆ cyclic group.

24. A process of paragraph 23 in which said 5 to 13-membered cyclic group is aromatic or heteroaromatic.

25. A process of paragraph 23 in which said 5 to 13-membered cyclic group is phenyl or a 6-membered heteroaromatic group.

26. A process of any of paragraphs 1 to 22 in which X is R⁶—(CH₂)ₚ—C(O)— or R⁶—(CH₂)ₚ—O—C(O)— and p is 0 or 1.

27. A process of any of paragraphs 1 to 22 in which X is benzyloxycarbonyl.

28. A process of any of paragraphs 1 to 14 in which the boronic acid is of formula (III)
X—(R)-Phe-(S)-Pro-Mpg-B(OH)₂ (III),
wherein X is as defined in any of paragraphs 1 and 23 to 27.

29. A process of any of paragraphs 1 to 14 in which the boronic species is Cbz-(R)-Phe-(S)-Pro-Mpg-BOPin.

30. A process of any of paragraphs 1 to 29 in which the boronic species in the starting solution comprises from 50% to 60% molecules having chiral centre C* of (R)-configuration and from 40% to 50% molecules having chiral centre C* of (S)-configuration.

31. A process of any of paragraphs 1 to 30 which further comprises converting the recovered precipitate to the acid of formula (I) by dissolving the precipitate in an organic solvent selected from halohydrocarbons and combinations thereof, agitating the resulting organic solution with an aqueous acid having a pH of below 3 whereby the dissolved precipitate is converted to the formula (I) acid, and recovering the formula (I) acid by evaporation.

32. A process of paragraph 31 in which the duration of contact between the organic solution and the aqueous acid is limited sufficiently to avoid substantial C—B bond breakage.

33. A process of paragraph 32 wherein the duration is not more than about 30 minutes when the contact takes place at room temperature.

34. A process of any of paragraphs 31 to 33 in which the aqueous acid is hydrochloric acid of about 2% w/v concentration or another aqueous mineral acid of similar pH.

35. A process of any of paragraphs 31 to 34 in which the organic solvent is selected from CH₂Cl₂ and CHCl₃.

36. A process of paragraph 35 in which the organic solvent is CH₂Cl₂.

37. A process of any of paragraphs 31 to 36 in which the formula (I) acid is dried.

38. A process of paragraph 37 in which the formula (I) acid is dried when it is in the organic solvent by contacting the solvent with a hygroscopic solid.

39. A process of any of paragraphs 31 to 38 in which the formula (I) acid, when in the organic solvent, is washed with an aqueous ammonium salt.

40. A process of any of paragraphs 31 to 39 in which organic solvent is evaporated from the recovered formula (I) acid.

41. A process of any of paragraphs 31 to 40 which further comprises converting the recovered acid of formula (I) to a pharmaceutically acceptable base addition salt thereof by dissolving the acid in acetonitrile, combining the resultant solution with an aqueous solution or suspension of a pharmaceutically acceptable base, and causing or allowing the base and the acid to react, then evaporating to dryness to obtain an evaporation residue.

42. A process of paragraph 41 in which the step of causing or allowing the acid and the base to react comprises agitating the combination of the acetonitrile solution of the acid and the aqueous solution or suspension of the base at a temperature of not more than 35° C.

43. A process of paragraph 42 in which the temperature is not more than 25° C.

44. A process of any of paragraphs 41 to 43 which further comprises:
(i) redissolving the evaporation residue in acetonitrile and evaporating the resulting solution to dryness; and
(ii) repeating step (i) as often as necessary to obtain a dry evaporation residue.

45. A process of paragraph 44 wherein the dry evaporation residue has a loss on drying of less than about 0.5% when determined by drying in a vacuum drier at 40° C. at 100 mbar for 2 hours.

46. A process of paragraph 44 which further comprises:
dissolving the dry evaporation residue in acetonitrile or tetrahydrofuran to form a solution;
adding, at a rate sufficiently slow to avoid lump formation, said solution to a 3:1 to 1:3 v/v mixture of diethylether and an aliphatic or cycloaliphatic solvent to form a precipitate, said solution being added to the diethylether/(cyclo)aliphatic solvent mixture in a ratio (solution:mixture) of from 1:5 to 1:15 v/v;
recovering the precipitate; and
removing solvent from the recovered precipitate under reduced pressure whilst maintaining the temperature at no more than 35° C.

47. A process of paragraph 46 wherein the solvent removal process is performed until the precipitate has a loss on drying of less than about 0.5% when determined by drying in a vacuum drier at 40° C. at 100 mbar for 2 hours.

48. A process of paragraph 46 or paragraph 47 in which the temperature at the start of the drying process is about 10° C. and is increased during the process to about 35° C.

49. A process of any of paragraphs 46 to 48 in which the aliphatic or cycloaliphatic solvent is n-heptane.

50. A process of any of paragraphs 41 to 49 in which the base comprises a cation of valency n and is used in a stoichiometry (boronic acid:base) of about n:1.

51. A process of any of paragraphs 41 to 50 in which the base is an alkali metal or alkaline earth metal base.

52. A process of any of paragraphs 41 to 50 in which the base is an alkali metal hydroxide.

53. A process of any of paragraphs 41 to 50 in which the base is sodium hydroxide.

54. A process of any of paragraphs 46 to 50 in which the base is sodium hydroxide and the dry evaporation residue is dissolved in acetonitrile.

55. A process of any of paragraphs 41 to 50 in which the base is an alkaline earth metal hydroxide.

56. A process of any of paragraphs 41 to 50 in which the base is calcium hydroxide.

57. A process of any of paragraphs 46 to 50 in which the base is calcium hydroxide and the dry evaporation residue is dissolved in tetrahydrofuran.

58. A process for hydrolysing an ester of a boronic acid as defined by any of paragraphs 1 and 15 to 29, comprising contacting the ester with an aqueous medium for a period sufficiently short substantially to avoid C—B bond cleavage.

59. A process of paragraph 58 wherein the aqueous medium is an aqueous acid having a pH of less than about 3.

60. A process of paragraph 59 wherein the aqueous acid is hydrochloric acid having a concentration of about 2% w/v or another aqueous mineral acid of similar pH.

61. A process of any of paragraphs 58 to 60 wherein the contact period is about 30 minutes or less.

62. A process of any of paragraphs 58 to 61 which is carried out at a temperature of about 25° C.±2° C.

63. A process of any of paragraphs 58 to 62 wherein the ester is a diethanolamine ester.

64. A process for making a pharmaceutically acceptable base addition salt of a boronic acid as defined by any of paragraphs 1 and 15 to 29, comprising:
dissolving the boronic acid in acetonitrile;
combining the resultant solution with an aqueous solution or suspension of a pharmaceutically acceptable base, and causing or allowing the base and the boronic acid to react;
evaporating to dryness to obtain an evaporation residue;
redissolving the evaporation residue in acetonitrile and evaporating the resulting solution to dryness; and
repeating the preceding step as often as necessary to obtain a substantially dry evaporation residue.

65. A process for making a boronic acid as defined in any of paragraphs 1 and 15 to 29 or a pharmaceutically acceptable base addition salt thereof, which boronic acid has an R¹ group of the formula —(CH₂)ₛ—O—R³ in which R³ is methyl or ethyl and s is independently 2, 3 or 4, comprising:
reacting a 1-metalloalkoxyalkane, where the alkoxyalkane is of the formula —(CH₂)ₛ—O—R³, and a borate ester, to form a compound of Formula (VI):
   (HO)₂B—(CH₂)ₛ—O—R³ (VI),
   and synthesising the boronic acid from the compound of Formula (VI) and, if desired, converting the acid into a said salt thereof.

66. A process of claim 65 wherein the synthesis of the boronic acid and, if it is the case, conversion into a salt thereof involves a process of any of paragraphs 1 to 64.

67. A compound selected from the group consisting of boronic acids of formula (I) as defined in any of paragraphs 1 and 15 to 29 and base addition salts thereof, the compound having a chiral purity of produced by a method of any of paragraphs 1 to 64.

68. A compound selected from the group consisting of boronic acids of formula (I) as defined in any of paragraphs 1 and 15 to 29 and base addition salts thereof, the compound having a diastereomeric excess over the (R,S,S) diastereomer of about 95% or more, optionally of about 99% or more, e.g. about 99.5% or more.

69. A salt selected from the monolithium, monosodium and monopotassium salts of Cbz-(R)-Phe-(S)-Pro-(R)-Mpg-B(OH)₂ and having a purity about that of the purity of such salt when prepared by the method of Example 2.

70. A salt of paragraph 69 having a diastereomeric excess of about 99.5% or more over the (R,S,S) diastereomer and a purity measured as % HPLC peak area of at least 97.5% when determined by the method of Example 5.

71. A salt of paragraph 69 or paragraph 70 which is the monosodium salt.

72. A salt selected form the hemicalcium and hemimagnesium salts of Cbz-(R)-Phe-(S)-Pro-(R)-Mpg-B(OH)₂ and having a purity about that of the purity of such salt when prepared by the method of Example 3.

73. A salt of claim 72 having a diastereomeric excess of about 99.5% or more and a purity measured as % HPLC peak area of at least 97.5% when determined by the method of Example 5.

74. A salt of claim 72 or claim 73 which is the hemicalcium salt.

75. A compound selected from Cbz-(R)-Phe-(S)-Pro-(R)-Mpg-B(OH)₂, and the esters and salts thereof (e.g. selected from the mono alkali metal salts and hemi alkaline earth metal salts), which compound has a purity measured as % HPLC peak area of at least 97.5%, for example 99% or more, e.g. 99.5% or more, the % HPLC peak area being determined by the method of Example 5.

76. A compound of paragraph 75 which is the monolithium, monosodium, hemicalcium or hemimagnesium salt.

77. A compound selected from boronic acids as defined in any of one of paragraphs 1 and 15 to 29 and the esters and salts thereof, the compound being substantially free of impurities detectable by HPLC, e.g. reverse phase HPLC.

78. A pharmaceutically acceptable base addition salt of a boronic acid as defined in any of paragraphs 1 and 15 to 29 which is substantially free of degradation products derived from cleavage of the C—B bond thereof.

79. A compound selected from boronic acids as defined in paragraph 65 and esters and salts of such acids, the compound being free of any compound which is of the same structure except for replacement of the R¹ group by a group of the formula —(CH₂)ₛ—H.

80. A pharmaceutically acceptable base addition salt of Cbz-(R)-Phe-(S)-Pro-(R)-Mpg-B(OH)₂ which is substantially free of the compound:

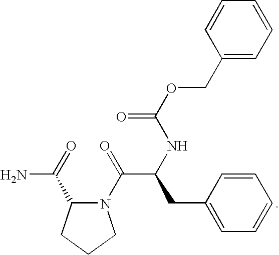

81. A salt of any of paragraphs 77 to 81 which is in an amount of at least 1 kg, e.g. has been produced in an amount of at least 100 kg a day.

82. A pharmaceutically acceptable base addition salt of a boronic acid as defined in any of paragraphs 1 and 15 to 29, for example Cbz-(R)-Phe-(S)-Pro-(R)-Mpg-B(OH)₂, which contains a trace amount of an aliphatic or cycloaliphatic solvent.

83. A salt of paragraph 81 or paragraph 82 wherein the solvent is an alkane.

84. A salt of paragraph 83 wherein the solvent is an n-alkane.

85. A salt of paragraph 84 wherein the solvent is n-heptane.

86. A salt of any of paragraphs 81 to 85 wherein the solvent is present in an amount of about 0.01% or less.

87. A product for use as a pharmaceutical, comprising a salt of any of paragraphs 67 to 86.

88. A pharmaceutical formulation comprising a salt of any of paragraphs 67 to 86 and a pharmaceutically acceptable diluent, excipient or carrier.

89. A pharmaceutical formulation in oral dosage form comprising a salt of any of paragraphs 72 to 74.

90. A pharmaceutical formulation of paragraph 89 which is adapted to release the salt in the duodenum.

91. A pharmaceutical formulation of paragraph 90 which is enterically coated.

92. A pharmaceutical formulation in parenteral dosage form comprising a salt of any of paragraphs 69 to 71.

93. A pharmaceutical formulation of paragraph 92 which is in dry particulate form for reconstitution.

94. A pharmaceutical formulation of paragraph 92 which is adapted for administration by infusion or injection, if necessary after reconstitution.

95. A pharmaceutical formulation of paragraph 94 which is an intravenous formulation.

96. A method of inhibiting thrombin in the treatment of disease comprising administering to a mammal a therapeutically effective amount of an active agent selected from the group consisting of the salts of any of paragraphs 67 to 86.

97. The use of a salt of any of paragraphs 67 to 86 for the manufacture of a medicament for treating thrombosis.

98. A method of treating venous and/or arterial thrombosis by prophylaxis or therapy, comprising administering to a mammal suffering from, or at risk of suffering from, thrombosis a therapeutically effective amount of a product selected from the salts of any of paragraphs 67 to 86.

99. A method of paragraph 98 in which the disease is an acute coronary syndrome.

100. A method of paragraph 98 in which the disease is acute myocardial infarction.

101. A method of paragraph 98 in which the disease is a venous thromboembolic event, selected from the group consisting of deep vein thrombosis and pulmonary embolism.

102. A method for preventing thrombosis in a haemodialysis circuit of a patient, comprising administering to the patient a therapeutically effective amount of a product selected from the salts of any of paragraphs 67 to 86.

103. A method for preventing a cardiovascular event in a patient with end stage renal disease, comprising administering to the patient a therapeutically effective amount of a product selected from the salts of any of paragraphs 67 to 86.

104. A method for preventing venous thromboembolic events in a patient receiving chemotherapy through an indwelling catheter, comprising administering to the patient a therapeutically effective amount of a product selected from the salts of any of paragraphs 67 to 86.

105. A method for preventing thromboembolic events in a patient undergoing a lower limb arterial reconstructive procedure, comprising administering to the patient a therapeutically effective amount of a product selected from the salts of any of paragraphs 67 to 86.

106. A method of inhibiting platelet procoagulant activity, comprising administering to a mammal at risk of, or suffering from, arterial thrombosis a therapeutically effective amount of a product selected from the salts of any of paragraphs 67 to 86.

107. A method of paragraph 106 in which the disease is an acute coronary syndrome.

108. A method of treating by way of therapy or prophylaxis an arterial disease selected from acute coronary syndromes, cerebrovascular thrombosis, peripheral arterial occlusion and arterial thrombosis resulting from atrial fibrillation, valvular heart disease, arterio-venous shunts, indwelling catheters or coronary stents, comprising administering to a mammal a therapeutically effective amount of a product selected from the salts of any of paragraphs 67 to 86.

109. A method of paragraph 108 in which the disease is an acute coronary syndrome.

110. The use of a salt of any of paragraphs 67 to 86 for the manufacture of a medicament for a treatment recited in any of paragraphs 104 to 109.

111. A pharmaceutical formulation comprising a combination of (i) a salt of any of paragraphs 67 to 86 and (ii) a further pharmaceutically active agent.

112. A pharmaceutical formulation comprising a combination of (i) a salt of any of paragraphs 67 to 86 and (ii) another cardiovascular treatment agent.

113. A formulation of paragraph 112 in which the other cardiovascular treatment agent comprises a lipid-lowering drug, a fibrate, niacin, a statin, a CETP inhibitor, a bile acid sequestrant, an anti-oxidant, a IIb/IIIa antagonist, an aldosterone inhibitor, an A2 antagonist, an A3 agonist, a beta-blocker, acetylsalicylic acid, a loop diuretic, an ACE inhibitor, an antithrombotic agent with a different mechanism of action, an antiplatelet agent, a thromboxane receptor and/or synthetase inhibitor, a fibrinogen receptor antagonist, a prostacyclin mimetic, a phosphodiesterase inhibitor, an ADP-receptor (P₂ T) antagonist, a thrombolytic, a cardioprotectant or a COX-2 inhibitor.

114. The use of a salt of any of paragraphs 67 to 86 for the manufacture of a medicament for treating, for example preventing, a cardiovascular disorder in co-administration with another pharmaceutically active agent.

115. The use of paragraph 89 in which the other active agent is another cardiovascular treatment agent.

116. The process of any of paragraphs 67 to 86, which further comprises formulating the salt into a pharmaceutical composition.

117. The use of diethanolamine to resolve by selective precipitation the (R,S,R) and (R,S,S) isomers of a boronic add of formula (I) as defined in any of paragraphs 1 and 15 to 29.

118. A product comprising an (R,S,R) isomer of a boronic acid of formula (I) as defined in any of paragraphs 1 and 15 to 29 whenever produced by a process which used diethanolamine to resolve the (R,S,R) and (R,S,S) isomers by precipitation.

119. A method for making an anti-thrombotic formulation, comprising making a salt of any of paragraphs 67 to 86 into such a formulation at a rate of at least 1000 kg of said salt a year.

120. A method for providing an antithrombotic formulation, comprising delivering to pharmacies a formulation of any of paragraphs 88 to 95 or 111 to 113.

121. A package comprising a pharmaceutical formulation containing a salt of any of paragraphs 67 to 86 and comprising a marketing authorisation number and/or a patient instruction leaflet.

122. A pharmaceutically acceptable base addition salt of Cbz-(R)-Phe-(S)-Pro-(R)-Mpg-B(OH)₂, which salt contains a trace amount of an aliphatic or cycloaliphatic solvent but is substantially free of an impurity of the structure:

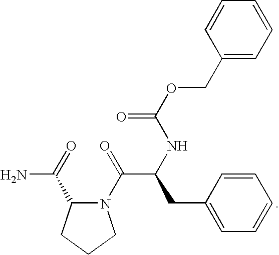

123. A process for making an aminoboronate of Formula (IX) 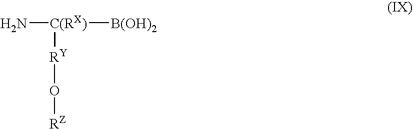
wherein
R^{X} is H or a substituent which does not prevent synthesis;
R^{Y} is alkylene; and
R^{Z} is alkyl,
the process comprising reacting a 1-metalloalkoxyalkane with a borate ester to form a boronic acid of the formula R^{Z}—O—R^{Y}—B(OH)₂, esterifying the acid, contacting the esterified acid with CH₂Cl₂ and ZnCl₂ in the presence of a strong base, contacting the resultant produce with LiHMDS and in turn contacting the resultant product with hydrogen chloride.

124. An aminoboronate of Formula (IX) of paragraph 123 which is free of contaminant of Formula (X):
H₂N—C(R^{X})(R^{Y})—B(OH)₂ (X).

125. A process for making an organoboronic acid of Formula (VII) 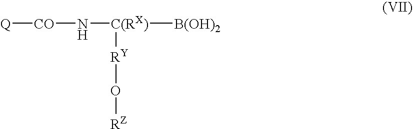
wherein
Q-CO comprises at least an amino acid residue;
R^{X} is H or a substituent which does not prevent synthesis;
R^{Y} is alkylene;
R^{Z} is alkyl,
the process comprising:
a) performing the method of paragraph 123 to make an aminoboronate of Formula (IX), or
b) providing an aminoboronate of paragraph 124, and reacting the aminoboronate with a compound selected from amino acids and peptides, which compounds are optionally N-terminally protected.

126. A compound selected from organoboronic acids of Formula (VII) as defined in paragraph 125, or an ester or salt thereof which is free of an impurity selected from compounds of Formula (VIII): 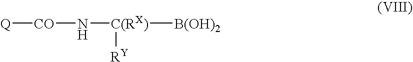
and esters and salts thereof.

127. A pharmaceutically acceptable base addition salt of a boronic acid of formula I as defined in any of paragraphs 1 and 15 to 29, wherein the boronic acid having the chiral centre C* of (R)-configuration is in a large diastereomeric excess.

128. A salt of paragraph 127 which is substantially free of any degradation product resulting from C—B bond cleavage.

129. A salt of paragraph 127 or paragraph 128 wherein R¹ is as defined in paragraph 65 and the salt is substantially free of the corresponding boronic acid species in which R¹ is —(CH₂)ₛH.

130. A pharmaceutical formulation comprising a salt of any of paragraphs 122-129.

131. A pharmaceutical formulation comprising a salt of any of paragraphs 67 to 86 and substantially free of any impurity derived from synthesis of the salt.

132. A compound selected from boronic acids of formula (Ia) and isolated from other stereoisomers of the compound: 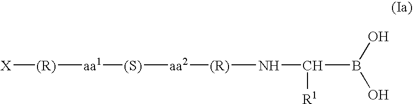
where:
X is H (to form NH₂) or an amino-protecting group;
aa¹ is an amino acid having a hydrocarbyl side chain containing no more than 20 carbon atoms and comprising at least one cyclic group having up to 13 carbon atoms;
aa² is an imino acid having from 4 to 6 ring members;
R⁹ is a straight chain alkyl group interrupted by one or more ether linkages and in which the total number of oxygen and carbon atoms is 3, 4, 5 or 6 or R⁹ is —(CH₂)ₘ—W where m is from 2, 3, 4 or 5 and W is —OH or halogen (F, Cl, Br or I).

133. A compound of paragraph 132 wherein aa¹ is selected from Dpa, Phe, Dcha and Cha.

134. A compound of paragraph 132 wherein aa¹ is Phe or Dpa, and optionally (IV), is Phe.

135. A compound of any of paragraphs 132 to134 wherein aa² is a residue of an imino acid of formula (IV) 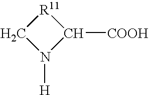
where R¹¹ is —CH₂—, —CH₂—CH₂—, —S—CH₂—, —S—C(CH₃)₂— or —CH₂—CH₂—CH₂—, which group, when the ring is 5- or 6-membered, is optionally substituted at one or more —CH₂— groups by from 1 to 3 C₁-C₃ alkyl groups, and optionally is Pro.

136. A compound of any of paragraphs 132 to135 wherein R¹ is a group of the formula —(CH₂)ₛ-Z, where s is 2, 3 or 4 and Z is —OH, —OMe, —OEt or halogen (F, Cl, Br or I), and optionally is selected from 2-bromoethyl, 2-chloroethyl, 2-methoxyethyl, 3-bromopropyl, 3-chloropropyl or 3-methoxypropyl, and optionally is 3-methoxypropyl.

137. A compound of paragraph 136 which of formula (VIII):
X—(R)-Phe-(S)-Pro(R)-Mpg-B(OH)₂ (VIII).

138. A compound of any of paragraphs 132 to 137 where X is R⁶—(CH₂)ₚ—C(O)—, R⁶—(CH₂)ₚ—S(O)₂—, R⁶—(CH₂)ₚ—NH—C(O)— or R⁶—(CH₂)ₚ—O—C(O)— wherein p is 0, 1, 2, 3, 4, 5 or 6 and R⁶ is H or a 5 to 13-membered cyclic group optionally substituted by 1, 2 or 3 substituents selected from halogen, amino, nitro, hydroxy, a C₅-C₆ cyclic group, C₁-C₄ alkyl and C₁-C₄ alkyl containing, and/or linked to the cyclic group through, an in-chain O, the aforesaid alkyl groups optionally being substituted by a substituent selected from halogen, amino, nitro, hydroxy and a C₅-C₆ cyclic group, and optionally wherein said 5 to 13-membered cyclic group is aromatic or heteroaromatic, e.g. is phenyl or a 6-membered heteroaromatic group.

139. A compound of any of paragraphs 132 to138 wherein X is R⁶—(CH₂)ₚ—C(O)— or R⁶—(CH₂)ₚ—O—C(O)— and p is 0 or 1, and optionally is benzyloxycarbonyl.

140. A compound of paragraph 132 which is Cbz-(R)-Phe-(S)-Pro-(R)-Mpg-B(OH)₂.

141. A compound of any of paragraphs 132 to 140 which is isolated.

142. A compound of any of paragraphs 132 to 141 which is in particulate form.

143. A compound of any of paragraphs 132 to143 which has a decrease in weight upon drying of less than 0.5% of its initial weight when dried in a vacuum drier at 40° C. at 100 mbar for 2 hours.

144. A compound of any of paragraphs 132 to 143 wherein the (R,S,R) isomer is in a diastereomeric excess of 95% or more over the (R,S,S) isomer.

145. A compound of paragraph 144 wherein the (R,S,R) isomer is in a diastereomeric excess of 99% or more, e.g. 99.5% or more.

146. A compound of any of paragraphs 132 to 145 which is substantially free of degradation product derived from cleavage of the C—B bond thereof.

147. A compound of paragraph 146 which has a purity measured as HPLC peak area of at least 97.5%, the % peak area being determined by the method of Example 43.

148. A compound of paragraph 147 wherein the purity is at least 99%, e.g. at least 99.5%.

149. A compound of any of paragraphs 146 to 148 which is Cbz-(R)-Phe-(S)-Pro-(R)-Mpg-B(OH)₂ and where the degradation product of which it is substantially free is

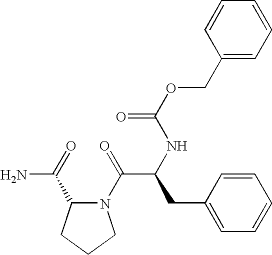

150. A compound of any of paragraphs 132 to149 in which R¹ is a group of the formula —(CH₂)ₛ-Z wherein Z is —OMe or —OEt and which is free of any compound which is of the same structure except for replacement of the R¹ group by a group of the formula —(CH₂)ₛ—H.

151. Cbz-(R)-Phe-(S)-Pro-(R)-Mpg-B(OH)₂ when in a diastereomeric excess of at least 99% over the corresponding (R,S,S) isomer, substantially free of the compound: 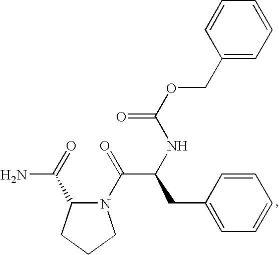
and free of the compound:

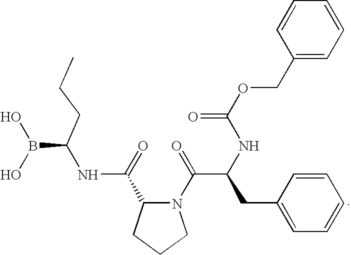

152. A product comprising isolated, dry Cbz-(R)-Phe-(S)-Pro-(R)-Mpg-B(OH)₂.

153. A product of paragraph 152 which is a particulate composition.

154. A product of paragraph 152 or paragraph 153 which is acceptable for pharmaceutical use, in particular sterile.

155. A process for making a pharmaceutically acceptable base addition salt of the compound of any of paragraphs 132 to134, by contacting the compound with a base capable of making such a salt.

156. The process of paragraph 155 wherein the salt is a metal salt, e.g. a salt of an alkali metal, an alkaline earth metal or zinc.

157. The process of paragraph 155 wherein the base is an organic base having a pKb of 7 or more, e.g. 7.5 or more, for example of 8 or more.

158. A product, wherein the product is an industrial product and it comprises a salt of any of paragraphs 67 to 86, 122 or 127 to 129, a formulation of any of paragraphs 111-113, boronic acid of any of paragraphs 67, 68, 77, 79, 126 or 132 to 154 or an ester of any of paragraphs 75, 77, 79 and 126.

159. A pharmaceutical formulation which comprises the following first and second species and optionally one or more other pharmaceutically acceptable components:
a) a therapeutically effective amount of a first species selected from (i) boronic acids of formula (IIa), (ii) boronate anions of the acid, (iii) any equilibrium form of (i) or (ii), and (iv) any combination of the aforegoing: 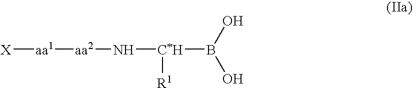
   where:
X is H (to form NH₂) or an amino-protecting group;
aa¹ is an amino acid of (R)-configuration having a hydrocarbyl side chain containing no more than 20 carbon atoms and comprising at least one cyclic group having up to 13 carbon atoms;
aa² is an imino acid of (S)-configuration having from 4 to 6 ring members;
C* is a chiral center of (R)-configuration;
R¹ is a group of the formula —(CH₂)ₛ-Z, where s is 2, 3 or 4 and Z is —OH, —OMe, —OEt or halogen selected from F, Cl, Br or I,
b) a second species selected from the group consisting of (v) pharmaceutically acceptable alkali metal ions, (vi) pharmaceutically acceptable basic organic nitrogen-containing compounds having a pKb of about 7 or more, (vii) any equilibrium form of (v), and (viii) any combination of the aforegoing,
   wherein the formulation is substantially free of degradation product derived from cleavage of the C—B bond of the first species.

160. A pharmaceutical formulation which comprises the following first and second species and optionally one or more other pharmaceutically acceptable components:
a) a therapeutically effective amount of a first species selected from (i) boronic acids of formula (IIa), (ii) boronate anions of the acid, (iii) any equilibrium form of (i) or (ii), and (iv) any combination of the aforegoing: 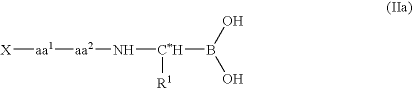
   where:
X is H (to form NH₂) or an amino-protecting group;
aa¹ is an amino acid of (R)-configuration having a hydrocarbyl side chain containing no more than 20 carbon atoms and comprising at least one cyclic group having up to 13 carbon atoms;
aa² is an imino acid of (S)-configuration having from 4 to 6 ring members;
C* is a chiral center of (R)-configuration;
R¹ is a group of the formula —(CH₂)₅-Z, where s is 2, 3 or 4 and Z is —OH, —OMe, —OEt or halogen selected from F, Cl, Br or I,
b) a second species selected from the group consisting of (v) pharmaceutically acceptable alkali metal ions, (vi) pharmaceutically acceptable basic organic nitrogen-containing compounds having a pKb of about 7 or more, (vii) any equilibrium form of (v), and (viii) any combination of the aforegoing,
   wherein the first species is in a diastereomeric excess of about 98% or more over the (R,S,S) diastereomer thereof.

161. A pharmaceutical formulation which comprises the following first and second species and optionally one or more other pharmaceutically acceptable components:
a) a therapeutically effective amount of a first species selected from (i) the boronic acid Cbz-(R)-Phe-(S)-Pro-(R)-Mpg-B(OH), (ii) boronate anions of the acid, (iii) any equilibrium form of (i) or (ii), and (iv) any combination of the aforegoing, and
b) a second species selected from the group consisting of (v) pharmaceutically acceptable alkali metal ions, (vi) pharmaceutically acceptable basic organic nitrogen-containing compounds having a pKb of about 7 or more, (vii) any equilibrium form of (v), and (viii) any combination of the aforegoing,
   wherein the formulation is substantially free of the following compound and equilibrium forms thereof:

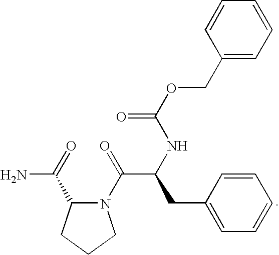

163. A pharmaceutical formulation which comprises the following first and second species and optionally one or more other pharmaceutically acceptable components:
a) a therapeutically effective amount of a first species selected from (i) the boronic acid Cbz-(R)-Phe-(S)-Pro-(R)-Mpg-B(OH), (ii) boronate anions of the acid, (iii) any equilibrium form of (i) or (ii), and (iv) any combination of the aforegoing, and
b) a second species selected from the group consisting of (v) pharmaceutically acceptable alkali metal ions, (vi) pharmaceutically acceptable basic organic nitrogen-containing compounds having a pKb of about 7 or more, (vii) any equilibrium form of (v), and (viii) any combination of the aforegoing,
   wherein the formulation is substantially free of the following compound and equilibrium forms thereof:

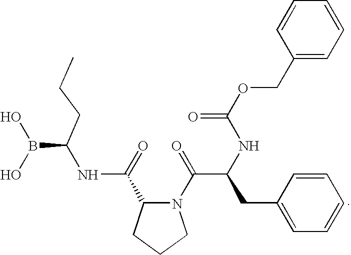

164. A pharmaceutical formulation which comprises the following first and second species and optionally one or more other pharmaceutically acceptable components:
a) a therapeutically effective amount of a first species selected from (i) the boronic acid Cbz-(R)-Phe-(S)-Pro-(R)-Mpg-B(OH), (ii) boronate anions of the acid, (iii) any equilibrium form of (i) or (ii), and (iv) any combination of the aforegoing, and
b) a second species selected from the group consisting of (v) pharmaceutically acceptable alkali metal ions, (vi) pharmaceutically acceptable basic organic nitrogen-containing compounds having a pKb of about 7 or more, (vii) any equilibrium form of (v), and (viii) any combination of the aforegoing,
   wherein the first species is in a diastereomeric excess of about 98% or more over the (R,S,S) diastereomer thereof.

1. A process for converting a diethanolamine adduct of a boronic acid of formula (I) below to the free acid of formula (I), the process comprising:
dissolving the adduct in an organic solvent selected from a halohydrocarbon or a mixture of halohydrocarbons;
agitating the resulting organic solution with an aqueous acid having a pH of below 3 whereby the dissolved adduct is converted to the formula (I) acid, the duration of contact between the organic solution and the aqueous acid being limited sufficiently to avoid substantial C—B bond breakage; and
recovering the formula (I) acid by evaporation,
formula (I) being: 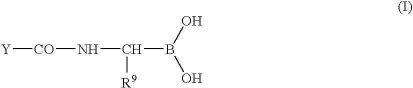
wherein
Y comprises a hydrophobic moiety which, together with the aminoboronic acid residue —NHCH(R⁹)—B(OH)₂, has affinity for the substrate binding site of thrombin; and
R⁹ is a straight chain alkyl group interrupted by one or more ether linkages and in which the total number of oxygen and carbon atoms is 3, 4, 5 or 6 or R⁹ is —(CH₂)ₘ—W where m is from 2, 3, 4 or 5 and W is —OH or halogen selected from F, Cl, Br or I.

2. The process of claim 1 wherein the contact takes place at room temperature and the duration is not more than about 30 minutes.

3. The process of claim 1 in which the organic solvent is selected from CH₂Cl₂ and CHCl₃.

4. The process of claim 1 in which the formula (I) acid is dried.

5. The process of claim 4 in which the formula (I) acid is dried when it is in the organic solvent by contacting the solvent with a hygroscopic solid.

6. A process of claim 1 in which the formula (I) acid, when in the organic solvent, is washed with an aqueous ammonium salt.

7. The process of claim 1 wherein R⁹ is an alkoxyalkyl group and Y is an optionally N-terminally protected dipeptide which binds to the S3 and S2 binding sites of thrombin and the peptide linkages in the acid are optionally and independently N-substituted by a C₁-C13 hydrocarbyl optionally containing in-chain or in-ring nitrogen, oxygen or sulfur and optionally substituted by a substituent selected from halo, hydroxy and trifluoromethyl.

8. The process of claim 7 wherein the S3-binding amino acid residue is of (R)-configuration, the S2-binding residue is of (S)-configuration, and the fragment —NHCH(R⁹)—B(OH) is of (R)-configuration.

9. The process of claim 1 wherein the boronic acid is of formula (II): 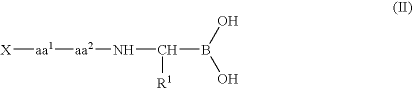
where:
X is H (to form NH₂) or an amino-protecting group;
aa¹ is an amino acid having a hydrocarbyl side chain containing no more than 20 carbon atoms and comprising at least one cyclic group having up to 13 carbon atoms;
aa² is an imino acid having from 4 to 6 ring members;
R¹ is a group of the formula —(CH₂)ₛ-Z, where s is 2, 3 or 4 and Z is —OH, —OMe, —OEt or halogen selected from F, Cl, Br or I.

10. The process of claim 9 wherein
X is R⁶—(CH₂)ₚ—C(O)—, R⁶—(CH₂)ₚ—S(O)₂—, R⁶—(CH₂)ₚ—NH—C(O)— or R⁶—(CH₂)ₚ—O—C(O)— wherein p is 0, 1, 2, 3, 4, 5 or 6 and R⁶ is H or a 5 to 13-membered cyclic group optionally substituted by 1, 2 or 3 substituents selected from halogen, amino, nitro, hydroxy, a C₅-C₆ cyclic group, C₁-C₄ alkyl, C₁-C₄ alkyl containing an in-chain O, a C₁-C₄ alkyl linked to the cyclic group through an in-chain O, or a C₁-C₄ alkyl both containing an in-chain O and linked to the cyclic group through an in-chain O, the aforesaid alkyl groups optionally being substituted by a substituent selected from halogen, amino, nitro, hydroxy and a C₅-C₆ cyclic group;
aa¹ is selected from Phe, Dpa and wholly or partially hydrogenated analogues thereof;
aa² is a residue of an imino acid of formula (IV) 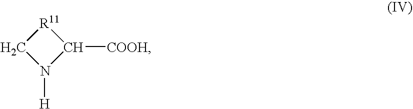
where R¹¹ is —CH₂—, —CH₂—CH₂—, —S—CH₂—, —S—C(CH₃)₂— or —CH₂—CH₂—CH₂—, which group, when the ring is 5- or 6-membered, is optionally substituted at one or more —CH₂— groups by from 1 to 3 C₁-C₃ alkyl groups;
R¹ is 2-bromoethyl, 2-chloroethyl, 2-methoxyethyl, 3-bromopropyl, 3-chloropropyl or 3-methoxypropyl.

11. The process of claim 10 wherein aa¹ is of (R)-configuration, aa² is of (S)-configuration and the fragment —NH—CH(R¹)—B(OH)₂ is of (R)-configuration.

12. The process of claim 1 in which the boronic acid of formula (I) is Cbz-(R)-Phe-(S)-Pro-(R)-Mpg-B(OH)₂.

13. The process of claim 11, wherein the process further comprises making the diethanolamine adduct in a process for separating diastereomers comprising:
combining in diethylether solution (A) a boronic species selected from the boronic acid (II) and its esters with alcohols, in which alcohols the sole potential electron donor heteroatoms are oxygens which, in the boronic ester, correspond to the oxygens of the ester functional group, the starting solution containing both boronic species having a fragment —NH—CH(R¹)—B(OH)₂ of (R) configuration and boronic species having a fragment —NH—CH(R¹)—B(OH)₂ of (S) configuration, and (B) diethanolamine, the diethanolamine being in an amount of 1.25±0.1 equivalents based on the boronic species in which fragment —NH—CH(R¹)—B(OH)₂ is of (R) configuration, and mixing to form a mixture;
causing or allowing the boronic species and the diethanolamine to react until a precipitate forms; and
recovering the precipitate that includes the diethanolamine adduct.

14. The process of claim 13 in which the diethanolamine is in an amount of about 1.25 equivalents based on the boronic species.

15. The process of claim 13 in which the alcohol is a diol which is not sterically hindered.

16. The process of claim 13 in which the alcohol is pinacol.

17. The process of claim 15 in which the recovered precipitate is washed with diethylether.

18. The process of claim 1 which further comprises converting the free acid of claim 1 to a pharmaceutically acceptable base addition salt thereof.

19. A boronic acid which is substantially free of degradation product derived from cleavage of the C—B bond thereof, formula (I) being: 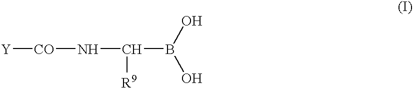
wherein
Y comprises a hydrophobic moiety which, together with the aminoboronic acid residue —NHCH(R⁹)—B(OH)₂, has affinity for the substrate binding site of thrombin; and
R⁹ is a straight chain alkyl group interrupted by one or more ether linkages and in which the total number of oxygen and carbon atoms is 3, 4, 5 or 6 or R⁹ is —(CH₂)ₘ—W where m is from 2, 3, 4 or 5 and W is —OH or halogen selected from F, Cl, Br or I.

20. The boronic acid of claim 19 which contains no more than about 0.5% of said degradation product by total weight.

21. The boronic acid of claim 19 which is of formula (IIa): 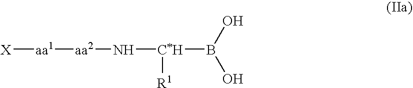
where:
X is H (to form NH₂) or an amino-protecting group;
aa¹ is an amino acid of (R)-configuration having a hydrocarbyl side chain containing no more than 20 carbon atoms and comprising at least one cyclic group having up to 13 carbon atoms;
aa² is an imino acid of (S)-configuration having from 4 to 6 ring members;
C* is a chiral center of (R)-configuration;
R¹ is a group of the formula —(CH₂)ₛ-Z, where s is 2, 3 or 4 and Z is —OH, —OMe, —OEt or halogen selected from F, Cl, Br or I,
and wherein the (R,S,R) isomer is in a diastereomeric excess of about 98% or more over the (R,S,S) isomer.

22. The boronic acid of claim 19 which is Cbz-Phe-Pro-Mpg-B(OH)₂ and it is substantially free of a degradation product of the following structure:

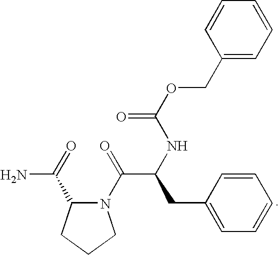

23. Cbz-(R)-Phe-(S)-Pro-(R)-Mpg-B(OH)₂ when in a diastereomeric excess of at least about 98% over the corresponding (R,S,S) isomer and substantially free of the compound:

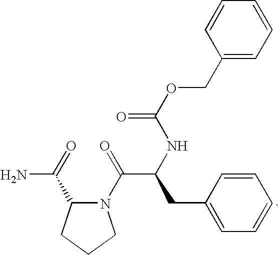

24. A process for separating diastereomers of a boronic acid of formula (I): 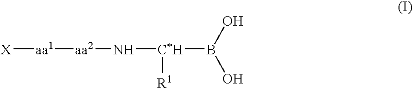
where:
X is H (to form NH₂) or an amino-protecting group;
aa¹ is an amino acid of (R) configuration selected from Phe, Dpa and wholly or partially hydrogenated analogues thereof;
aa² is an imino acid of (S) configuration having from 4 to 6 ring members;
R¹ is a group of the formula —(CH₂)₅-Z, where s is 2, 3 or 4 and Z is —OH, —OMe, —OEt or halogen selected from F, Cl, Br or I,
and where C* is a chiral centre,
the process comprising:
combining in diethylether solution (A) a boronic species selected from the boronic acid (I) and its esters, the boronic species including molecules having a chiral centre C* of (R) configuration and molecules having a chiral centre C* of (S) configuration, and (B) diethanolamine, the diethanolamine being in an amount of about 1.25±0.1 equivalents based on the boronic species in which the chiral centre C* is of (R) configuration;
causing or allowing the boronic species and the diethanolamine to react until a precipitate forms; and
recovering the precipitate that includes the diethanolamine adduct.

25. A process of claim 24 in which the diethanolamine is in an amount of about 1.25 equivalents based on the boronic species.

26. The process of claim 24 in which the alcohol is a diol which is not sterically hindered.

27. A process for making a pharmaceutically acceptable base addition salt of a boronic acid of formula (I) below, comprising:
dissolving the boronic acid in acetonitrile;
combining the resultant solution with an aqueous solution or suspension of a pharmaceutically acceptable base, and causing or allowing the base and the boronic acid to react;
evaporating to dryness to obtain an evaporation residue;
redissolving the evaporation residue in acetonitrile and evaporating the resulting solution to dryness; and
repeating the preceding step as often as necessary to obtain a substantially dry evaporation residue, formula (I) being: 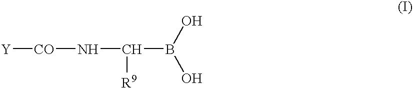
wherein
Y comprises a hydrophobic moiety which, together with the aminoboronic acid residue —NHCH(R⁹)—B(OH)₂, has affinity for the substrate binding site of thrombin; and
R⁹ is a straight chain alkyl group interrupted by one or more ether linkages and in which the total number of oxygen and carbon atoms is 3, 4, 5 or 6 or R⁹ is —(CH₂)ₘ—W where m is from 2, 3, 4 or 5 and W is —OH or halogen selected from F, Cl, Br or I.

28. A compound selected from the group consisting of diethanolamine esters and pharmaceutically acceptable base addition salts of boronic acids of formula (I), 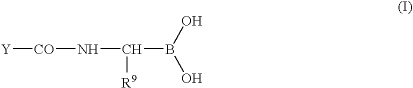
wherein
Y comprises a hydrophobic moiety which, together with the aminoboronic acid residue —NHCH(R⁹)—B(OH)₂, has affinity for the substrate binding site of thrombin, wherein Y is an optionally N-terminally protected dipeptide which binds to the S3 and S2 binding sites of thrombin and the peptide linkages in the acid are optionally and independently N-substituted by a C₁-C₁₃ hydrocarbyl optionally containing in-chain or in-ring nitrogen, oxygen or sulfur and optionally substituted by a substituent selected from halo, hydroxy and trifluoromethyl and wherein the S3-binding amino acid residue is of (R)-configuration, the S2-binding residue is of (S)-configuration, and the fragment —NHCH(R⁹)—B(OH) is of (R)-configuration; and
R⁹ is a straight chain alkyl group interrupted by one or more ether linkages and in which the total number of oxygen and carbon atoms is 3, 4, 5 or 6 or R⁹ is —(CH₂)ₘ—W where m is from 2, 3, 4 or 5 and W is —OH or halogen selected from F, Cl, Br or I,
and wherein the (R,S,R) isomer is present in a diastereomeric excess of about 98% or more over the (R,S,S) isomer.

29. A compound of claim 28 which is a salt of a metal or of an organic nitrogen-containing compound having a pKb of about 7 or more.

30. A compound selected from the group consisting of diethanolamine esters and pharmaceutically acceptable base addition salts of boronic acids of formula (IIIa) and in a diastereomeric excess of about 99% or more over the (R,S,S) isomer of the compound: 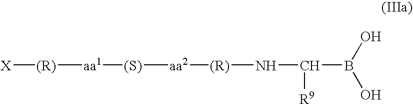
where:
X is H (to form NH₂) or an amino-protecting group;
aa¹ is an amino acid having a hydrocarbyl side chain containing no more than 20 carbon atoms and comprising at least one cyclic group having up to 13 carbon atoms;
aa² is an imino acid having from 4 to 6 ring members;
R⁹ is a straight chain alkyl group interrupted by one or more ether linkages and in which the total number of oxygen and carbon atoms is 3, 4, 5 or 6 or R⁹ is —(CH₂)ₘ—W where m is from 2, 3, 4 or 5 and W is —OH or halogen (F, Cl, Br or I).

31. A compound of claim 30 wherein:
aa¹ is selected from Dpa, Phe, Dcha and Cha.;
aa² is a residue of an imino acid of formula (IV) 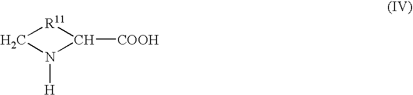
where R¹¹ is —CH₂—, —CH₂—CH₂—, —S—CH₂—, —S—C(CH₃)₂— or —CH₂—CH₂—CH₂—, which group, when the ring is 5- or 6-membered, is optionally substituted at one or more —CH₂— groups by from 1 to 3 C₁-C₃ alkyl groups;
R¹ is a group of the formula —(CH₂)ₛ-Z, where s is 2, 3 or 4 and Z is —OH, —OMe, —OEt or halogen selected from F, Cl, Br or I.

32. A compound of claim 30 wherein the acid is of formula (VIII):
X—(R)-Phe-(S)-Pro-(R)-Mpg-B(OH)₂ (VIII).

33. A compound of claim 30 which is a said salt.

34. A compound of claim 31 which is a salt of an alkali metal, an alkaline earth metal or zinc.

35. A pharmaceutically acceptable base addition salt of a boronic acid of formula (I) below which contains a trace amount of an aliphatic or cycloaliphatic solvent, formula (I) being: 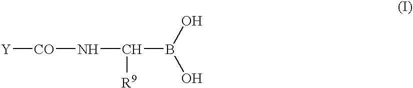
wherein
Y comprises a hydrophobic moiety which, together with the aminoboronic acid residue —NHCH(R⁹)—B(OH)₂, has affinity for the substrate binding site of thrombin; and
R⁹ is a straight chain alkyl group interrupted by one or more ether linkages and in which the total number of oxygen and carbon atoms is 3, 4, 5 or 6 or R⁹ is —(CH₂)ₘ—W where m is from 2, 3, 4 or 5 and W is —OH or halogen selected from F, Cl, Br or I.

36. A pharmaceutical formulation which comprises the following first and second species and optionally one or more other pharmaceutically acceptable components:
a) a therapeutically effective amount of a first species selected from (i) boronic acids of formula (IIa), (ii) boronate anions of the acid, (iii) any equilibrium form of (i) or (ii), and (iv) any combination of the aforegoing: 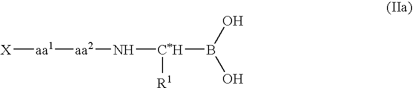
where:
X is H (to form NH₂) or an amino-protecting group;
aa¹ is an amino acid of (R)-configuration having a hydrocarbyl side chain containing no more than 20 carbon atoms and comprising at least one cyclic group having up to 13 carbon atoms;
aa² is an imino acid of (S)-configuration having from 4 to 6 ring members;
C* is a chiral center of (R)-configuration;
R¹ is a group of the formula —CH₂)ₛ-Z, where s is 2, 3 or 4 and Z is —OH, —OMe, —OEt or halogen selected from F, Cl, Br or I,
b) a second species selected from the group consisting of (v) pharmaceutically acceptable alkali metal ions, (vi) pharmaceutically acceptable basic organic nitrogen-containing compounds having a pKb of about 7 or more, (vii) any equilibrium form of (v), and (viii) any combination of the aforegoing,
wherein the formulation is substantially free of degradation product derived from cleavage of the C—B bond of the first species.
